SEQUENCE LISTING (13 sequences)

SEQ ID NO: 1 - protein, 594 aa, Artificial Sequence
  Synthetic Construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 2 - protein, 340 aa, Artificial Sequence
  Synthetic Construct
MGKSKEISQDLRKKIVDLHKSGSSLGAISKRLKVPRSSVQTIVRKYKHHGTTQPSYRSGRRRYLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 3 - protein, 340 aa, Artificial Sequence
  Synthetic Construct
MGKSKEISQDLRKRIVDLHKSGSSLGAISKRLAVPRSSVQTIVRKYKHHGTTQPSYRSGRRRYLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 4 - protein, 1053 aa, Artificial Sequence
  Synthetic Construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - protein, 88 aa, Homo sapiens
PAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 6 - protein, 90 aa, Homo sapiens
MLPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 7 - DNA, 270 bp, Homo sapiens
atgctgcctgcaccaaagaacctggtggtgtctcatgtgacagaggatagtgccagactgtcatggactgctcccgacgcagccttcgatagttttatcatcgtgtaccgggagaacatcgaaaccggcgaggccattgtcctgacagtgccagggtccgaacgctcttatgacctgacagatctgaagcccggaactgagtactatgtgcagatcgccggcgtcaaaggaggcaatatcagcttccctctgtccgcaatcttcaccaca

SEQ ID NO: 8 - protein, 4 aa, Homo sapiens
TEDS

SEQ ID NO: 9 - protein, 7 aa, Homo sapiens
TAPDAAF

SEQ ID NO: 10 - protein, 6 aa, Homo sapiens
SEKVGE

SEQ ID NO: 11 - protein, 4 aa, Homo sapiens
GSER

SEQ ID NO: 12 - protein, 5 aa, Homo sapiens
GLKPG

SEQ ID NO: 13 - protein, 7 aa, Homo sapiens
KGGHRSN

(19) United States
(12) Patent Application Publication
(10) Pub. No.: US 2019/0185880 A1
(43) Pub. Date: Jun. 20, 2019

(54) TRANSPOSON SYSTEM AND METHODS OF USE

(71) Applicant: Poseida Therapeutics, Inc., San Diego, CA (US)

(72) Inventors: Devon SHEDLOCK, San Diego, CA (US); David HERMANSON, San Diego, CA (US); Eric OSTERTAG, Lexington, KY (US)

(21) Appl. No.: 15/849,545

(22) Filed: Dec. 20, 2017

(51) Int. Cl. C12N 15/85; C12N 15/87; C12N 9/12; C12N 13/00; C12N 5/0783; C07K 14/78; C07K 16/00; C12N 9/22; A61K 35/17

(52) U.S. Cl. CPC C12N 15/85; C12N 15/87; C12N 9/1241; C12N 13/00; C12N 5/0636; C07K 14/78; C12N 2800/80; C12N 9/22; A61K 35/17; C12N 2800/90; C12N 2501/2307; C12N 2501/2315; C07K 16/00

(57) ABSTRACT

Disclosed are methods for the ex-vivo genetic modification of an immune cell comprising delivering to the immune cell, (a) a nucleic acid or amino acid sequence comprising a sequence encoding a transposase enzyme and (b) a recombinant and non-naturally occurring DNA sequence comprising a DNA sequence encoding a transposon.

### RELATED APPLICATIONS

This application is a Continuation Application of International Application No. PCT/US2017/019531 filed on Feb. 24, 2017, which claims priority to U.S. Patent Application No. 62/300,387, filed Feb. 26, 2016, the contents of which are each herein incorporated by reference in their entirety.

### INCORPORATION OF SEQUENCE LISTING

The contents of the text file named “POTH-007-C01US_SeqList.txt”, which was created on Dec. 20, 2017 and is 23 KB in size, are hereby incorporated by reference in their entirety.

### FIELD OF THE DISCLOSURE

The present invention is directed to compositions and methods for targeted gene modification.

### BACKGROUND

Ex vivo genetic modification of non-transformed primary human T lymphocytes using non-viral vector-based gene transfer delivery systems has been extremely difficult. As a result, most groups have generally used viral vector-based transduction such as retrovirus, including lentivirus. A number of non-viral methods have been tested and include antibody-targeted liposomes, nanoparticles, aptamer siRNA chimeras, electroporation, nucleofection, lipofection, and peptide transduction. Overall, these approaches have resulted in poor transfection efficiency, direct cell toxicity, or a lack of experimental throughput.

The use of plasmid vectors for genetic modification of human lymphocytes has been limited by low efficiency using currently available plasmid transfection systems and by the toxicity that many plasmid transfection reagents have on these cells. There is a long-felt and unmet need for a method of nonviral gene modification in immune cells.

### SUMMARY

When compared with viral transduction of immune cells, such as T lymphocytes, delivery of transgenes via DNA transposons, such as piggyBac and Sleeping Beauty, offers significant advantages in ease of use, ability to delivery much larger cargo, speed to clinic and cost of production. The piggyBac DNA transposon, in particular, offers additional advantages in giving long-term, high-level and stable expression of transgenes, and in being significantly less mutagenic than a retrovirus, being non-oncogenic and being fully reversible. Previous attempts to use DNA transposons to deliver transgenes to T cells have been unsuccessful at generating commercially viable products or manufacturing methods because the previous methods have been inefficient. For example, the poor efficiency demonstrated by previous methods of using DNA transposons to deliver transgenes to T cells has resulted in the need for prolonged expansion ex vivo. Previous unsuccessful attempts by others to solve this problem have all focused on increasing the amount of DNA transposon delivered to the immune cell, which has been a strategy that worked well for non-immune cells. This disclosure demonstrates that increasing the amount of DNA transposon makes the efficiency problem worse in immune cells by increasing DNA-mediated toxicity. To solve this problem, counterintuitively, the methods of the disclosure decrease the amount of DNA delivered to the immune cell. Using the methods of the disclosure, the data provided herein demonstrate not only that decreasing the amount of DNA transposon introduced into the cell increased viability but also that this method increased the percentage of cells that harbored a transposition event, resulting in a viable commercial process and a viable commercial product. Thus, the methods of the disclosure demonstrate success where others have failed.

The disclosure provides a nonviral method for the ex-vivo genetic modification of an immune cell comprising delivering to the immune cell, (a) a nucleic acid or amino acid sequence comprising a sequence encoding a transposase enzyme and (b) a recombinant and non-naturally occurring DNA sequence comprising a DNA sequence encoding a transposon. In certain embodiments, the method further comprises the step of stimulating the immune cell with one or more cytokine(s).

In certain embodiments of the methods of the disclosure, the sequence encoding a transposase enzyme is an mRNA sequence. The mRNA sequence encoding a transposase enzyme may be produced in vitro.

In certain embodiments of the methods of the disclosure, the sequence encoding a transposase enzyme is a DNA sequence. The DNA sequence encoding a transposase enzyme may be produced in vitro. The DNA sequence may be a cDNA sequence.

In certain embodiments of the methods of the disclosure, the sequence encoding a transposase enzyme is an amino acid sequence. The amino acid sequence encoding a transposase enzyme may be produced in vitro. A protein sPBo may be delivered following pre-incubation with transposon DNA.

In certain embodiments of the methods of the disclosure, the delivering step comprises electroporation or nucleofection of the immune cell.

In certain embodiments of the methods of the disclosure, the step of stimulating the immune cell with one or more cytokine(s) occurs following the delivering step. Alternatively, or in addition, in certain embodiments, the step of stimulating the immune cell with one or more cytokine(s) occurs prior to the delivering step. In certain embodiments, the one or more cytokine(s) comprise(s) IL-2, IL-21, IL-7 and/or IL-15.

In certain embodiments of the methods of the disclosure, the immune cell is an autologous immune cell. The immune cell may be a human immune cell and/or an autologous immune cell. The immune cell may be derived from a non-autologous source, including, but not limited to a primary cell, a cultured cell or cell line, an embryonic or adult stem cell, an induced pluripotent stem cell or a transdifferentiated cell. The immune cell may have been previously genetically modified or derived from a cell or cell line that has been genetically modified. The immune cell may be modified or may be derived from a cell or cell line that has been modified to suppress one or more apoptotic pathways. The immune cell may be modified or may be derived from a cell or cell line that has been modified to be “universally” allogenic by a majority of recipients in the context, for example, of a therapy involving an adoptive cell transfer.

In certain embodiments of the methods of the disclosure, the immune cell is an activated immune cell.

In certain embodiments of the methods of the disclosure, the immune cell is an resting immune cell.

In certain embodiments of the methods of the disclosure, the immune cell is a T-lymphocyte. In certain embodiments, the T-lymphocyte is an activated T-lymphocyte. In certain embodiments, the T-lymphocyte is a resting T-lymphocyte.

In certain embodiments of the methods of the disclosure, the immune cell is a Natural Killer (NK) cell.

In certain embodiments of the methods of the disclosure, the immune cell is a Cytokine-induced Killer (CIK) cell.

In certain embodiments of the methods of the disclosure, the immune cell is a Natural Killer T (NKT) cell.

In certain embodiments of the methods of the disclosure, the immune cell is isolated or derived from a human.

In certain embodiments of the methods of the disclosure, the immune cell is isolated or derived from a non-human mammal. In certain embodiments, the non-human mammal is a rodent, a rabbit, a cat, a dog, a pig, a horse, a cow, or a camel. In certain embodiments, the immune cell is isolated or derived from a non-human primate.

In certain embodiments of the methods of the disclosure, the transposase enzyme is a Super piggyBac™ (sPBo) transposase enzyme. The Super piggyBac (PB) transposase enzyme may comprise or consist of an amino acid sequence at least 75% identical to:

(SEQ ID NO: 1)

MGSSLDDEHILSALLQSDDELVGEDSDSEVSDHVSEDDVQSDTEEAFIDE
VHEVQPTSSGSEILDEQNVIEQPGSSLASNRILTLPQRTIRGKNKHCWST
SKSTRRSRVSALNIVRSQRGPTRMCRNIYDPLLCFKLFFTDEIISEIVKW
TNAEISLKRRESMTSATFRDTNEDEIYAFFGILVMTAVRKDNHMSTDDLF
DRSLSMVYVSVMSRDRFDFLIRCLRMDDKSIRPTLRENDVFTPVRKIWDL
FIHQCIQNYTPGAHLTIDEQLLGFRGRCPFRVYIPNKPSKYGIKILMMCD
SGTKYMINGMPYLGRGTQTNGVPLGEYYVKELSKPVHGSCRNITCDNWFT
SIPLAKNLLQEPYKLTIVGTVRSNKREIPEVLKNSRSRPVGTSMFCFDGP
LTLVSYKPKPAKMVYLLSSCDEDASINESTGKPQMVMYYNQTKGGVDTLD
QMCSVMTCSRKTNRWPMALLYGMINIACINSFIIYSHNVSSKGEKVQSRK
KFMRNLYMSLTSSFMRKRLEAPTLKRYLRDNISNILPKEVPGTSDDSTEE
PVMKKRTYCTYCPSKIRRKANASCKKCKKVICREHNIDMCQSCF.

In certain embodiments of the methods of the disclosure, the transposase enzyme is a Sleeping Beauty transposase enzyme (see, for example, U.S. Pat. No. 9,228,180, the contents of which are incorporated herein in their entirety). In certain embodiments, the Sleeping Beauty transposase is a hyperactive Sleeping Beauty SB100X transposase. In certain embodiments, the Sleeping Beauty transposase enzyme comprises an amino acid sequence at least 75% identical to:

(SEQ ID NO: 2)

MGKSKEISQDLRKKIVDLHKSGSSLGAISKRLKVPRSSVQTIVRKYKHHG
TTQPSYRSGRRRYLSPRDERTLVRKVQINPRTTAKDLVKMLEETGTKVSI
STVKRVLYRHNLKGRSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMRKENYVDILKQHLKTSVRKLKLGRKWVFQMDNDPKHT
SKVVAKWLKDNKVKVLEWPSQSPDLNPIENLWAELKKRVRARRPTNLTQL
HQLCQEEWAKIHPTYCGKLVEGYPKRLTQVKQFKGNATKY.

In certain embodiments, including those wherein the Sleeping Beauty transposase is a hyperactive Sleeping Beauty SB100X transposase, the Sleeping Beauty transposase enzyme comprises an amino acid sequence at least 75% identical to:

(SEQ ID NO: 3)

MGKSKEISQDLRKRIVDLHKSGSSLGAISKRLAVPRSSVQTIVRKYKHHG
TTQPSYRSGRRRYLSPRDERTLVRKVQINPRTTAKDLVKMLEETGTKVSI
STVKRVLYRHNLKGHSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMDAVQYVDILKQHLKTSVRKLKLGRKWVFQHDNDPKHT
SKVVAKWLKDNKVKVLEWPSQSPDLNPIENLWAELKKRVRARRPTNLTQL
HQLCQEEWAKIHPNYCGKLVEGYPKRLTQVKQFKGNATKY.

In certain embodiments of the methods of the disclosure, the recombinant and non-naturally occurring DNA sequence comprising a DNA sequence encoding a transposon may be circular. As a nonlimiting example, the DNA sequence encoding a transposon may be a plasmid vector. As a nonlimiting example, the DNA sequence encoding a transposon may be a minicircle DNA vector.

In certain embodiments of the methods of the disclosure, the recombinant and non-naturally occurring DNA sequence encoding a transposon may be linear. The linear recombinant and non-naturally occurring DNA sequence encoding a transposon may be produced in vitro. Linear recombinant and non-naturally occurring DNA sequences of the disclosure may be a product of a restriction digest of a circular DNA. In certain embodiments, the circular DNA is a plasmid vector or a minicircle DNA vector. Linear recombinant and non-naturally occurring DNA sequences of the disclosure may be a product of a polymerase chain reaction (PCR). Linear recombinant and non-naturally occurring DNA sequences of the disclosure may be a double-stranded Doggybone™ DNA sequence. Doggybone™ DNA sequences of the disclosure may be produced by an enzymatic process that solely encodes an antigen expression cassette, comprising antigen, promoter, poly-A tail and telomeric ends.

In certain embodiments of the methods of the disclosure, the recombinant and non-naturally occurring DNA sequence encoding a transposon further comprises a sequence encoding a chimeric antigen receptor or a portion thereof. Chimeric antigen receptors (CARs) of the disclosure may comprise (a) an ectodomain comprising an antigen recognition region, (b) a transmembrane domain, and (c) an endodomain comprising at least one costimulatory domain. In certain embodiments, the ectodomain may further comprise a signal peptide. Alternatively, or in addition, in certain embodiments, the ectodomain may further comprise a hinge between the antigen recognition region and the transmembrane domain. In certain embodiments of the CARs of the disclosure, the signal peptide may comprise a sequence encoding a human CD2, CD3δ, CD3ε, CD3γ, CD3ζ, CD4, CD8α, CD19, CD28, 4-1BB or GM-CSFR signal peptide. In certain embodiments of the CARs of the disclosure, the signal peptide may comprise a sequence encoding a human CD8α signal peptide. In certain embodiments, the transmembrane domain may comprise a sequence encoding a human CD2, CD3δ, CD3ε, CD3γ, CD3ζ, CD4, CD8α, CD19, CD28, 4-1BB or GM-CSFR transmembrane domain. In certain embodiments of the CARs of the disclosure, the transmembrane domain may comprise a sequence encoding a human CD8α transmembrane domain. In certain embodiments of the CARs of the disclosure, the endodomain may comprise a human CD3ζ endodomain. In certain embodiments of the CARs of the disclosure, the at least one costimulatory domain may comprise a human 4-1BB, CD28, CD40, ICOS, MyD88, OX-40 intracellular segment, or any combination thereof. In certain embodiments of the CARs of the disclosure, the at least one costimulatory domain may comprise a CD28 and/or a 4-1BB costimulatory domain. In certain embodiments of the CARs of the disclosure, the hinge may comprise a sequence derived from a human CD8α, IgG4, and/or CD4 sequence. In certain embodiments of the CARs of the disclosure, the hinge may comprise a sequence derived from a human CD8α sequence.

In certain embodiments of the methods of the disclosure, the recombinant and non-naturally occurring DNA sequence encoding a transposon further comprises a sequence encoding a chimeric antigen receptor or a portion thereof. The portion of the sequence encoding a chimeric antigen receptor may encode an antigen recognition region. The antigen recognition region may comprise one or more complementarity determining region(s). The antigen recognition region may comprise an antibody, an antibody mimetic, a protein scaffold or a fragment thereof. In certain embodiments, the antibody is a chimeric antibody, a recombinant antibody, a humanized antibody or a human antibody. In certain embodiments, the antibody is affinity-tuned. Nonlimiting examples of antibodies of the disclosure include a single-chain variable fragment (scFv), a VHH, a single domain antibody (sdAB), a small modular immunopharmaceutical (SMIP) molecule, or a nanobody. In certain embodiments, the VHH is camelid. Alternatively, or in addition, in certain embodiments, the VHH is humanized. Nonlimiting examples of antibody fragments of the disclosure include a complementary determining region, a variable region, a heavy chain, a light chain, or any combination thereof. Nonlimiting examples of antibody mimetics of the disclosure include an affibody, an afflilin, an affimer, an affitin, an alphabody, an anticalin, and avimer, a DARPin, a Fynomer, a Kunitz domain peptide, or a monobody. Nonlimiting examples of protein scaffolds of the disclosure include a Centyrin.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 10.0 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 100 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 7.5 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 75 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 6.0 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 60 μg/mL. In certain embodiments, the transposase is a Sleeping Beauty transposase. In certain embodiments, the Sleeping Beauty transposase is a Sleeping Beauty 100X (SB100X) transposase.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 5.0 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 50 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 2.5 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 25 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 1.67 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 16.7 μg/mL. In certain embodiments, the transposase is a Super piggyBac (PB) transposase.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 0.55 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 5.5 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 0.19 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 1.9 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 0.10 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 1.0 μg/mL.

In certain embodiments of the methods of the disclosure, the nucleic acid sequence encoding the transposase enzyme is a RNA sequence, and an amount of the DNA sequence encoding the transposon is equal to or less than 10.0 μg per 100 μL of an electroporation or nucleofection reaction. In certain embodiments, a concentration of the amount of the DNA sequence encoding the transposon in the electroporation or nucleofection reaction is equal to or less than 100 μg/mL.

In certain embodiments of the methods of the disclosure, the nucleic acid sequence encoding the transposase enzyme is a RNA sequence, and an amount of the DNA sequence encoding the transposon is equal to or less than 7.5 μg per 100 μL of an electroporation or nucleofection reaction. In certain embodiments, a concentration of the amount of the DNA sequence encoding the transposon in the electroporation or nucleofection reaction is equal to or less than 75 μg/mL.

In certain embodiments of the methods of the disclosure, the nucleic acid sequence encoding the transposase enzyme is a RNA sequence, and an amount of the DNA sequence encoding the transposon is equal to or less than 6.0 μg per 100 μL of an electroporation or nucleofection reaction. In certain embodiments, a concentration of the amount of the DNA sequence encoding the transposon in the electroporation or nucleofection reaction is equal to or less than 60 μg/mL. In certain embodiments, the transposase is a Sleeping Beauty transposase. In certain embodiments, the Sleeping Beauty transposase is a Sleeping Beauty 100X (SB100X) transposase.

In certain embodiments of the methods of the disclosure, the nucleic acid sequence encoding the transposase enzyme is a RNA sequence, and an amount of the DNA sequence encoding the transposon is equal to or less than 5.0 μg per 100 μL of an electroporation or nucleofection reaction. In certain embodiments, a concentration of the amount of the DNA sequence encoding the transposon in the electroporation or nucleofection reaction is equal to or less than 50 μg/mL.

In certain embodiments of the methods of the disclosure, the nucleic acid sequence encoding the transposase enzyme is a RNA sequence, and an amount of the DNA sequence encoding the transposon is equal to or less than 2.5 μg per 100 μL of an electroporation or nucleofection reaction. In certain embodiments, a concentration of the amount of the DNA sequence encoding the transposon in the electroporation or nucleofection reaction is equal to or less than 25 μg/mL.

In certain embodiments of the methods of the disclosure, the nucleic acid sequence encoding the transposase enzyme is a RNA sequence, and an amount of the DNA sequence encoding the transposon is equal to or less than 1.67 μg per 100 μL of an electroporation or nucleofection reaction. In certain embodiments, a concentration of the amount of the DNA sequence encoding the transposon in the electroporation or nucleofection reaction is equal to or less than 16.7 μg/mL. In certain embodiments, the transposase is a Super piggyBac (PB) transposase.

In certain embodiments of the methods of the disclosure, the nucleic acid sequence encoding the transposase enzyme is a RNA sequence, and an amount of the DNA sequence encoding the transposon is equal to or less than 0.55 μg per 100 μL of an electroporation or nucleofection reaction. In certain embodiments, a concentration of the amount of the DNA sequence encoding the transposon in the electroporation or nucleofection reaction is equal to or less than 5.5 μg/mL.

In certain embodiments of the methods of the disclosure, the nucleic acid sequence encoding the transposase enzyme is a RNA sequence, and an amount of the DNA sequence encoding the transposon is equal to or less than 0.19 μg per 100 μL of an electroporation or nucleofection reaction. In certain embodiments, a concentration of the amount of the DNA sequence encoding the transposon in the electroporation or nucleofection reaction is equal to or less than 1.9 μg/mL.

In certain embodiments of the methods of the disclosure, the nucleic acid sequence encoding the transposase enzyme is a RNA sequence, and an amount of the DNA sequence encoding the transposon is equal to or less than 1.0 μg per 100 μL of an electroporation or nucleofection reaction. In certain embodiments, a concentration of the amount of the DNA sequence encoding the transposon in the electroporation or nucleofection reaction is equal to or less than 1.0 μg/mL.

The disclosure provides an immune cell modified according to the method of the disclosure. The immune cell may be a T-lymphocyte, a Natural Killer (NK) cell, a Cytokine-induced Killer (CIK) cell or a Natural Killer T (NKT) cell. The immune cell may be further modified by a second gene editing tool, including, but not limited to those gene editing tools comprising an endonuclease operably-linked to either a Cas9 or a TALE sequence. In certain embodiments of the second gene editing tool, the endonuclease is operably-linked to either a Cas9 or a TALE sequence covalently. In certain embodiments of the second gene editing tool, the endonuclease is operably-linked to either a Cas9 or a TALE sequence non-covalently. In certain embodiments, the Cas9 is an inactivated Cas9 (dCas9). In certain embodiments, the inactivated Cas9 comprises D10A and N580A within the catalytic site. In certain embodiments, the Cas9 is a small and inactivated Cas9 (dSaCas9). In certain embodiments, the dSaCas9 comprises the amino acid sequence of

| (SEQ ID NO: 4) | |
|---|---|
| 1 | mkrnyilglA igitsvgygi idyetrdvid agvrlfkean vennegrrsk rgarrlkrrr |
| 61 | rhriqrvkkl lfdynlltdh selsginpye arvkglsqkl seeefsaall hlakrrgvhn |
| 121 | vneveedtgn elstkeqisr nskaleekyv aelqlerlkk dgevrgsinr fktsdyvkea |
| 181 | kqllkvqkay hqldqsfidt yidlletrrt yyegpgegsp fgwkdikewy emlmghctyf |
| 241 | peelrsvkya ynadlynaln dlnnlvitrd enekleyyek fqiienvfkq kkkptlkqia |
| 301 | keilvneedi kgyrvtstgk peftnlkvyh dikditarke iienaelldq iakiltiyqs |
| 361 | sediqeeltn lnseltqeei eqisnlkgyt gthnlslkai nlildelwht ndnqiaifnr |
| 421 | lklvpkkvdl sqqkeipttl vddfilspvv krsfiqsikv inaiikkygl pndiiielar |
| 481 | eknskdaqkm inemqkrnrq tnerieeiir ttgkenakyl iekiklhdmq egkclyslea |
| 541 | ipledllnnp fnyevdhiip rsvsfdnsfn nkvlvkqeeA skkgnrtpfq ylsssdskis |
| 601 | yetfkkhiln lakgkgrisk tkkeylleer dinrfsvqkd finrnlvdtr yatrglmnll |
| 661 | rsyfrvnnld vkvksinggf tsflrrkwkf kkernkgykh haedaliian adfifkewkk |
| 721 | ldkakkvmen qmfeekqaes mpeieteqey keifitphqi khikdfkdyk yshrvdkkpn |
| 781 | relindtlys trkddkgntl ivnnlnglyd kdndklkkli nkspekllmy hhdpqtyqkl |
| 841 | klimeqygde knplykyyee tgnyltkysk kdngpvikki kyygnklnah lditddypns |
| 901 | rnkvvklslk pyrfdvyldn gvykfvtvkn ldvikkenyy evnskcyeea kklkkisnqa |
| 961 | efiasfynnd likingelyr vigvnndlln rievnmidit yreylenmnd krppriikti |
| 1021 | asktqsikky stdilgnlye vkskkhpqii kkg. |

The disclosure provides an immune cell modified according to the method of the disclosure. The immune cell may be a T-lymphocyte, a Natural Killer (NK) cell, a Cytokine-induced Killer (CIK) cell or a Natural Killer T (NKT) cell. The immune cell may be further modified by a second gene editing tool, including, but not limited to those gene editing tools comprising an endonuclease operably-linked to either a Cas9 or a TALE sequence. Alternatively or in addition, the second gene editing tool may include an excision-only piggyBac transposase to re-excise the inserted sequences or any portion thereof. For example, the excision-only piggyBac transposase may be used to “re-excise” the transposon.

The disclosure provides a composition comprising the immune cell of the disclosure.

The disclosure provides a use of a composition comprising the immune cell of the disclosure for the treatment of a disease or disorder in a subject in need thereof. In certain embodiments, the disease or disorder is a cancer. In certain embodiments, the disease or disorder is an infectious disease. For example, the infectious disease may be caused by a virus, bacterium, yeast, microbe or any combination thereof. In certain embodiments, the immune cell of the composition is autologous. In certain embodiments, the immune cell of the composition is allogeneic.

The disclosure provides a culture media for enhancing viability of a modified immune cell comprising IL-2, IL-21, IL-7, IL-15 or any combination thereof. The modified immune cell may be a T-lymphocyte, a Natural Killer (NK) cell, a Cytokine-induced Killer (CIK) cell or a Natural Killer T (NKT) cell. The modified immune cell may contain one or more exogenous DNA sequences. The modified immune cell may contain one or more exogenous RNA sequences. The modified immune cell may have been electroporated or nucleofected.

### BRIEF DESCRIPTION OF THE DRAWINGS

FIG. 1 is a series of graphs depicting transfection efficiency and cell viability following plasmid DNA nucleofection in primary human T lymphocytes.

FIG. 2 is a series of graphs depicting DNA cytotoxicity to T cells.

FIG. 3 is a series of graphs showing that DNA-mediated cytotoxicity in T cells is dose dependent.

FIG. 4 is a series of graphs showing that extracellular plasmid DNA is not cytotoxic.

FIG. 5 is a series of graphs depicting efficient transposition using sPBo mRNA in Jurkat cells.

FIG. 6 is a series of graphs depicting efficient transposition in T lymphocytes using sPBo mRNA

FIG. 7 is a series of graphs depicting efficient delivery of linearized DNA transposon products.

FIG. 8 is a series of graphs showing that addition of that IL-7 and IL-15 and immediate stimulation of T cells post-nucleofection enhances cell viability.

FIG. 9 is a series of graphs showing that IL-7 and IL-15 rescue T cells from DNA mediated toxicity

FIG. 10 is a series of graphs showing that immediate stimulation of T cells post-nucleofection enhances cell viability.

FIG. 11A-C is a series of graphs depicting T cell transposition with varying amounts of DNA. Primary human pan T cells were nucleofected with varying amounts of DNA using piggyBac™. T cells were nucleofected with the indicated amounts of transposon and 5 μg sPBo mRNA. Cells were then stimulated on day 2 post-nucleofection through CD3 and CD28. As expected, T cells nucleofected with high amounts of DNA exhibited high episomal expression at day 1 post nucleofection whereas almost no episomal expression was observed at low DNA doses. In contrast, following expansion at day 21 post nucleofection the greatest percentage of transgene positive cells were observed in lower DNA amounts peaking at 1.67 μg for this transposon. (A) Flow analysis for transgene positive cells at day 1 and 21. (B) Percentage of transgene positive T cells. (C) Percentage of viable T cells at day 1 and 21. For all graphs shown in this figure, the Y-axis ranges from 0 to 100% in increments of 20% and the X-axis ranges from 0 to 10⁵ by powers of 10.

FIG. 12A-B is a series of graphs depicting T cell transposition with low DNA amounts using the Sleeping Beauty™ 100X (SB100X) transposase. Primary human pan T cells were nucleofected with GFP plasmids encoding either the piggyBac™ (PB) or Sleeping Beauty™ (SB) ITRs. (A) Cells were nucleofected with the indicated amounts of SB transposon and 1 μg SB transposase mRNA. (B) Cells were nucleofected with the indicated amounts of SB transposase and 0.75 μg SB transposon. Flow analysis was performed on day 14 post nucleofection for all samples. For all graphs shown in this figure, the Y-axis ranges from 0 to 250K in increments of 50K and the X-axis ranges from 0 to 10⁵ by powers of 10.

### DETAILED DESCRIPTION

Disclosed are compositions and methods for the ex-vivo genetic modification of an immune cell comprising delivering to the immune cell, (a) a nucleic acid or amino acid sequence comprising a sequence encoding a transposase enzyme and (b) a recombinant and non-naturally occurring DNA sequence comprising a DNA sequence encoding a transposon. In certain embodiments, the method further comprises the step of stimulating the immune cell with one or more cytokine(s).

Centyrins of the disclosure may comprise a protein scaffold, wherein the scaffold is capable of specifically binding an antigen. Centyrins of the disclosure may comprise a protein scaffold comprising a consensus sequence of at least one fibronectin type III (FN3) domain, wherein the scaffold is capable of specifically binding an antigen. The at least one fibronectin type III (FN3) domain may be derived from a human protein. The human protein may be Tenascin-C. The consensus sequence may comprise LPAPKNLVVSEVTEDSLRLSWTAPDAAFDSFLIQYQESEKVGEAINLTVPGSERSYDL TGLKPGTEYTVSIYGVKGGHRSNPLSAEFTT (SEQ ID NO: 5) or MLPAPKNLVVSEVTEDSLRLSWTAPDAAFDSFLIQYQESEKVGEAINLTVPGSERSYD LTGLKPGTEYTVSIYGVKGGHRSNPLSAEFTT (SEQ ID NO: 6). The consensus sequence may comprise an amino sequence at least 74% identical to LPAPKNLVVSEVTEDSLRLSWTAPDAAFDSFLIQYQESEKVGEAINLTVPGSERSYDL TGLKPGTEYTVSIYGVKGGHRSNPLSAEFTT (SEQ ID NO: 5) or MLPAPKNLVVSEVTEDSLRLSWTAPDAAFDSFLIQYQESEKVGEAINLTVPGSERSYD LTGLKPGTEYTVSIYGVKGGHRSNPLSAEFTT (SEQ ID NO: 6). The consensus sequence may encoded by a nucleic acid sequence comprising atgctgcctgcaccaaagaacctggtggtgtctcatgtgacagaggatagtgccagactgtcatggactgctcccgacgcagccttcg atagttttatcatcgtgtaccgggagaacatcgaaaccggcgaggccattgtcctgacagtgccagggtccgaacgctcttatgacctg acagatctgaagcccggaactgagtactatgtgcagatcgccggcgtcaaaggaggcaatatcagcttccctctgtccgcaatcttcac caca (SEQ ID NO: 7). The consensus sequence may be modified at one or more positions within (a) a A-B loop comprising or consisting of the amino acid residues TEDS (SEQ ID NO: 8) at positions 13-16 of the consensus sequence; (b) a B-C loop comprising or consisting of the amino acid residues TAPDAAF (SEQ ID NO: 9) at positions 22-28 of the consensus sequence; (c) a C-D loop comprising or consisting of the amino acid residues SEKVGE (SEQ ID NO: 10) at positions 38-43 of the consensus sequence; (d) a D-E loop comprising or consisting of the amino acid residues GSER (SEQ ID NO: 11) at positions 51-54 of the consensus sequence; (e) a E-F loop comprising or consisting of the amino acid residues GLKPG (SEQ ID NO: 12) at positions 60-64 of the consensus sequence; (f) a F-G loop comprising or consisting of the amino acid residues KGGHRSN (SEQ ID NO: 13) at positions 75-81 of the consensus sequence; or (g) any combination of (a)-(f). Centyrins of the disclosure may comprise a consensus sequence of at least 5 fibronectin type III (FN3) domains, at least 10 fibronectin type III (FN3) domains or at least 15 fibronectin type III (FN3) domains. The scaffold may bind an antigen with at least one affinity selected from a K_{D} of less than or equal to 10⁻⁹M, less than or equal to 10⁻¹⁰M, less than or equal to 10⁻¹¹M, less than or equal to 10⁻¹²M, less than or equal to 10⁻¹³M, less than or equal to 10⁻¹⁴M, and less than or equal to 10⁻¹⁵M. The K_{D} may be determined by surface plasmon resonance.

The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g. resistance to enzymatic degradation), and lower production costs. Exemplary antibody mimetics include, but are not limited to, an affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody.

Affibody molecules of the disclosure comprise a protein scaffold comprising or consisting of one or more alpha helix without any disulfide bridges. Preferably, affibody molecules of the disclosure comprise or consist of three alpha helices. For example, an affibody molecule of the disclosure may comprise an immunoglobulin binding domain. An affibody molecule of the disclosure may comprise the Z domain of protein A.

Affilin molecules of the disclosure comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly-folded protein molecule are considered exposed amino acids. Any one or more of these exposed amino acids may be modified to specifically bind to a target sequence or antigen.

Affimer molecules of the disclosure comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

Affitin molecules of the disclosure comprise an artificial protein scaffold, the structure of which may be derived, for example, from a DNA binding protein (e.g. the DNA binding protein Sac7d). Affitins of the disclosure selectively bind a target sequence, which may be the entirety or part of an antigen. Exemplary affitins of the disclosure are manufactured by randomizing one or more amino acid sequences on the binding surface of a DNA binding protein and subjecting the resultant protein to ribosome display and selection. Target sequences of affitins of the disclosure may be found, for example, in the genome or on the surface of a peptide, protein, virus, or bacteria. In certain embodiments of the disclosure, an affitin molecule may be used as a specific inhibitor of an enzyme. Affitin molecules of the disclosure may include heat-resistant proteins or derivatives thereof.

Alphabody molecules of the disclosure may also be referred to as Cell-Penetrating Alphabodies (CPAB). Alphabody molecules of the disclosure comprise small proteins (typically of less than 10 kDa) that bind to a variety of target sequences (including antigens). Alphabody molecules are capable of reaching and binding to intracellular target sequences. Structurally, alphabody molecules of the disclosure comprise an artificial sequence forming single chain alpha helix (similar to naturally occurring coiled-coil structures). Alphabody molecules of the disclosure may comprise a protein scaffold comprising one or more amino acids that are modified to specifically bind target proteins. Regardless of the binding specificity of the molecule, alphabody molecules of the disclosure maintain correct folding and thermostability.

Anticalin molecules of the disclosure comprise artificial proteins that bind to target sequences or sites in either proteins or small molecules. Anticalin molecules of the disclosure may comprise an artificial protein derived from a human lipocalin. Anticalin molecules of the disclosure may be used in place of, for example, monoclonal antibodies or fragments thereof. Anticalin molecules may demonstrate superior tissue penetration and thermostability than monoclonal antibodies or fragments thereof. Exemplary anticalin molecules of the disclosure may comprise about 180 amino acids, having a mass of approximately 20 kDa. Structurally, anticalin molecules of the disclosure comprise a barrel structure comprising antiparallel beta-strands pairwise connected by loops and an attached alpha helix. In preferred embodiments, anticalin molecules of the disclosure comprise a barrel structure comprising eight antiparallel beta-strands pairwise connected by loops and an attached alpha helix.

Avimer molecules of the disclosure comprise an artificial protein that specifically binds to a target sequence (which may also be an antigen). Avimers of the disclosure may recognize multiple binding sites within the same target or within distinct targets. When an avimer of the disclosure recognize more than one target, the avimer mimics function of a bi-specific antibody. The artificial protein avimer may comprise two or more peptide sequences of approximately 30-35 amino acids each. These peptides may be connected via one or more linker peptides. Amino acid sequences of one or more of the peptides of the avimer may be derived from an A domain of a membrane receptor. Avimers have a rigid structure that may optionally comprise disulfide bonds and/or calcium. Avimers of the disclosure may demonstrate greater heat stability compared to an antibody.

DARPins (Designed Ankyrin Repeat Proteins) of the disclosure comprise genetically-engineered, recombinant, or chimeric proteins having high specificity and high affinity for a target sequence. In certain embodiments, DARPins of the disclosure are derived from ankyrin proteins and, optionally, comprise at least three repeat motifs (also referred to as repetitive structural units) of the ankyrin protein. Ankyrin proteins mediate high-affinity protein-protein interactions. DARPins of the disclosure comprise a large target interaction surface.

Fynomers of the disclosure comprise small binding proteins (about 7 kDa) derived from the human Fyn SH3 domain and engineered to bind to target sequences and molecules with equal affinity and equal specificity as an antibody.

Kunitz domain peptides of the disclosure comprise a protein scaffold comprising a Kunitz domain. Kunitz domains comprise an active site for inhibiting protease activity. Structurally, Kunitz domains of the disclosure comprise a disulfide-rich alpha+beta fold. This structure is exemplified by the bovine pancreatic trypsin inhibitor. Kunitz domain peptides recognize specific protein structures and serve as competitive protease inhibitors. Kunitz domains of the disclosure may comprise Ecallantide (derived from a human lipoprotein-associated coagulation inhibitor (LACI)).

Monobodies of the disclosure are small proteins (comprising about 94 amino acids and having a mass of about 10 kDa) comparable in size to a single chain antibody. These genetically engineered proteins specifically bind target sequences including antigens. Monobodies of the disclosure may specifically target one or more distinct proteins or target sequences. In preferred embodiments, monobodies of the disclosure comprise a protein scaffold mimicking the structure of human fibronectin, and more preferably, mimicking the structure of the tenth extracellular type III domain of fibronectin. The tenth extracellular type III domain of fibronectin, as well as a monobody mimetic thereof, contains seven beta sheets forming a barrel and three exposed loops on each side corresponding to the three complementarity determining regions (CDRs) of an antibody. In contrast to the structure of the variable domain of an antibody, a monobody lacks any binding site for metal ions as well as a central disulfide bond. Multispecific monobodies may be optimized by modifying the loops BC and FG. Monobodies of the disclosure may comprise an adnectin.

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the invention.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to one embodiment hereof, the term “antibody hereof” in its broadest sense also covers such analogs. Generally, in such analogs, one or more amino acid residues may have been replaced, deleted and/or added, compared to the antibodies hereof as defined herein.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) which generally refers to an antibody fragment having a single monomeric variable antibody domain, (for example, from camelids). Such antibody fragment types will be readily understood by a person having ordinary skill in the art.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Embodiments defined by each of these transition terms are within the scope of this invention.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In certain embodiments, a specific binding interaction will discriminate between desirable and undesirable antigens in a sample, in some embodiments more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some embodiments, fusion polypeptides and/or nucleic acids encoding such fusion polypeptides include conservative substitutions have been introduced by modification of polynucleotides encoding polypeptides of the invention. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the plant the polynucleotide construct in such a manner that the construct gains access to the interior of the host cell. The methods of the invention do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

By “stable transformation” is intended that the polynucleotide construct introduced into a plant integrates into the genome of the host and is capable of being inherited by progeny thereof. By “transient transformation” is intended that a polynucleotide construct introduced into the host does not integrate into the genome of the host.

As used throughout the disclosure, the term “genetically modified plant (or transgenic plant)” refers to a plant which comprises within its genome an exogenous polynucleotide. Generally, and preferably, the exogenous polynucleotide is stably integrated into the genome such that the polynucleotide is passed on to successive generations. The exogenous polynucleotide may be integrated into the genome alone or as part of a recombinant expression cassette. “Transgenic” is used herein to include any cell, cell line, callus, tissue, plant part or plant, the genotype of which has been altered by the presence of exogenous nucleic acid including those trans genies initially so altered as well as those created by sexual crosses or asexual propagation from the initial transgenic. The term “transgenic” as used herein does not encompass the alteration of the genome (chromosomal or extrachromosomal) by conventional plant breeding methods or by naturally occurring events such as random cross-fertilization, non-recombinant viral infection, non-recombinant bacterial transformation, non-recombinant transposition, or spontaneous mutation.

As used throughout the disclosure, the term “modifying” is intended to mean that the sequence is considered modified simply by the binding of the polypeptide. It is not intended to suggest that the sequence of nucleotides is changed, although such changes (and others) could ensue following binding of the polypeptide to the nucleic acid of interest. In some embodiments, the nucleic acid sequence is DNA. Modification of the nucleic acid of interest (in the sense of binding thereto by a polypeptide modified to contain modular repeat units) could be detected in any of a number of methods (e.g. gel mobility shift assays, use of labelled polypeptides—labels could include radioactive, fluorescent, enzyme or biotin/streptavidin labels). Modification of the nucleic acid sequence of interest (and detection thereof) may be all that is required (e.g. in diagnosis of disease). Desirably, however, further processing of the sample is performed. Conveniently the polypeptide (and nucleic acid sequences specifically bound thereto) is separated from the rest of the sample. Advantageously the polypeptide-DNA complex is bound to a solid phase support, to facilitate such separation. For example, the polypeptide may be present in an acrylamide or agarose gel matrix or, more preferably, is immobilized on the surface of a membrane or in the wells of a microtitre plate.

All percentages and ratios are calculated by weight unless otherwise indicated.

All percentages and ratios are calculated based on the total composition unless otherwise indicated.

Every maximum numerical limitation given throughout this disclosure includes every lower numerical limitation, as if such lower numerical limitations were expressly written herein. Every minimum numerical limitation given throughout this disclosure will include every higher numerical limitation, as if such higher numerical limitations were expressly written herein. Every numerical range given throughout this disclosure will include every narrower numerical range that falls within such broader numerical range, as if such narrower numerical ranges were all expressly written herein.

The values disclosed herein are not to be understood as being strictly limited to the exact numerical values recited. Instead, unless otherwise specified, each such value is intended to mean both the recited value and a functionally equivalent range surrounding that value. For example, a value disclosed as “20 μm” is intended to mean “about 20 μm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Example 1: Ex Vivo Genetic Modification of T Cells

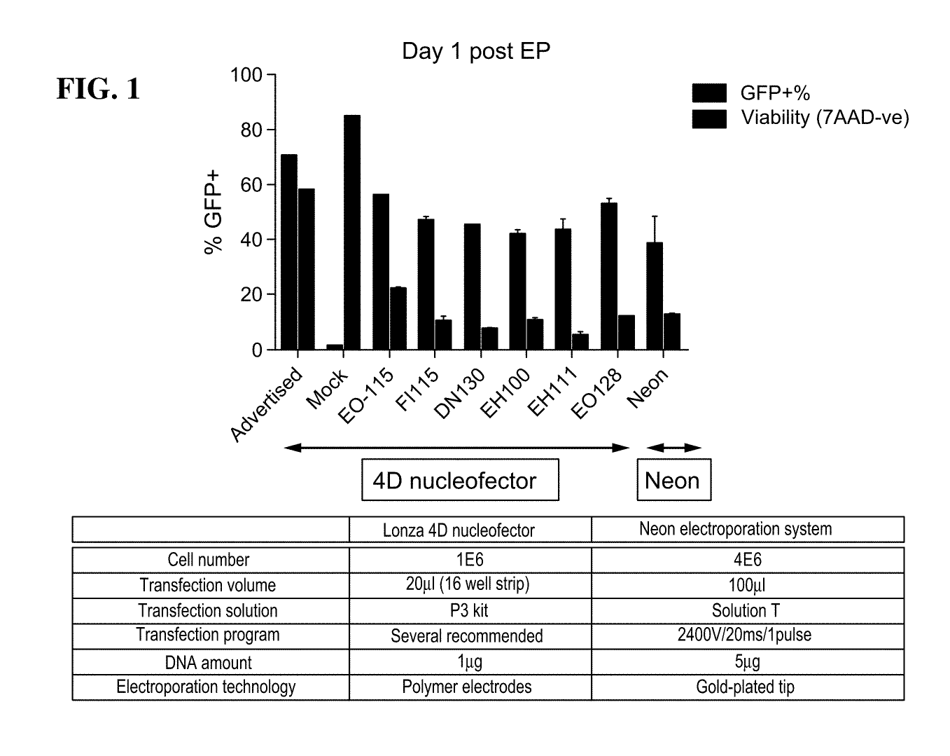

The piggyBac™ (PB) transposon system was used for genetically modifying human lymphocytes for production of autologous CAR-T immunotherapies and other applications. T Lymphocytes purified from patient blood or apheresis product was electroporated with a plasmid DNA transposon and a transposase. Several different electroporation systems have been used for T cell delivery of the transposon system, including the Neon (Thermo Fisher), BTX ECM 830 (Harvard Apparatus), Gene Pulser (BioRad), MaxCyte PulseAgile (MaxCyte), and the Amaxa 2B and Amaxa 4D (Lonza). Some were tested using manufacturer provided or recommended electroporation buffer, as well as several in-house developed buffers. Results were consistent with the prevailing dogma that resting T lymphocytes are particularly refractory to DNA transfection and that there appeared to be an inverse relationship between electroporation efficiency, as measured by GFP expression from the electroporated plasmid, and cell viability. FIG. 1 shows an example of an experiment testing multiple electroporation systems and nucleofection programs.

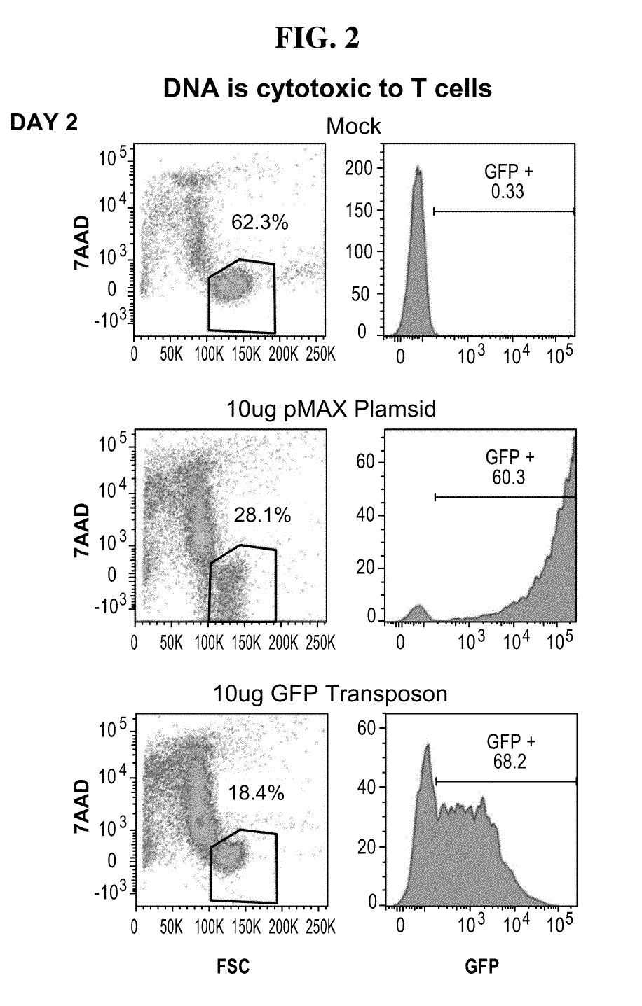

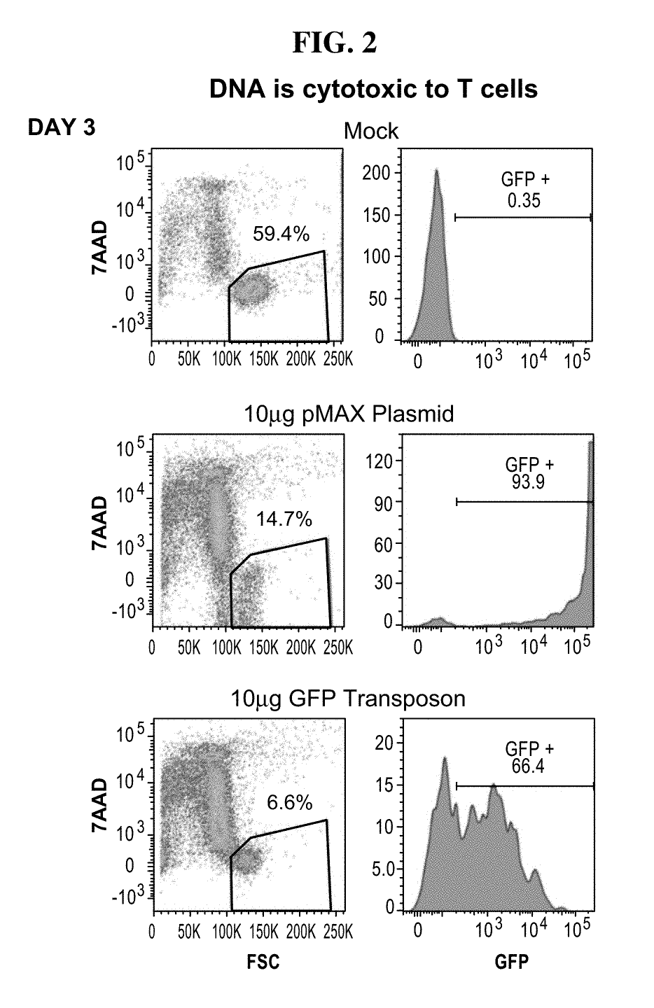

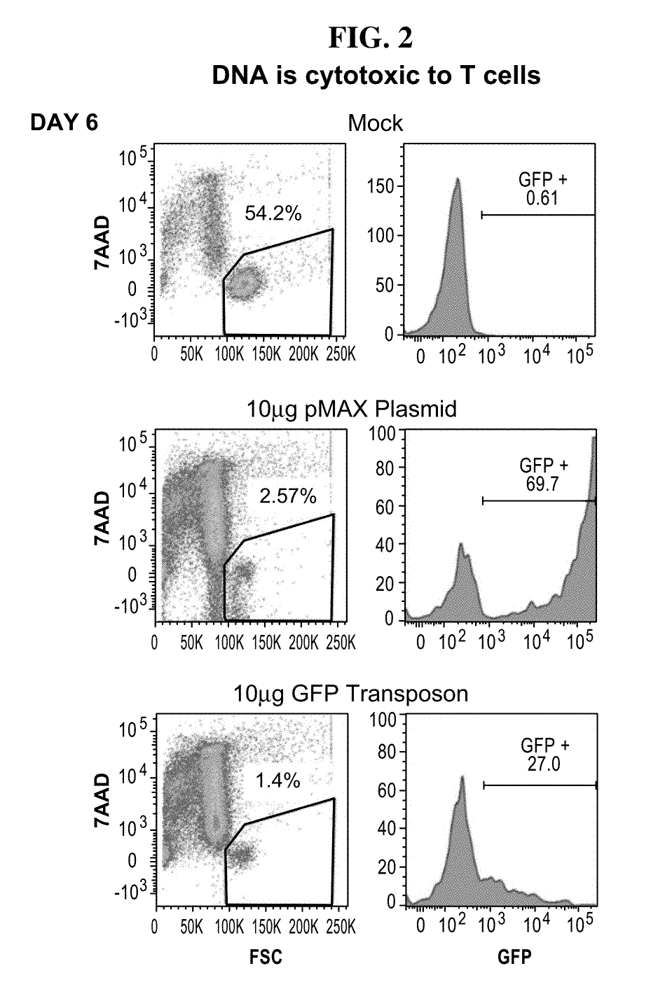

To further test whether or not plasmid DNA was toxic to T cells during nucleofection, primary human T lymphocytes were electroporated with two different DNA plasmids. The first plasmid was a pmaxGFP™ plasmid that is provided as a control plasmid in the Lonza Amaxa nucleofection kit. It is highly purified by HPLC and does not contain endotoxin at detectable levels. The second plasmid was our in-house produced PB transposon encoding a human EF1 alpha promoter driving GFP. Transfection efficiency, as measured by GFP expression from the electroporated plasmid, and cell viability was assessed by FACS at days 2, 3, and 6 post-electroporation. Data are displayed in FIG. 2. While mock electroporated cells (no plasmid DNA) exhibited relatively high levels of cell viability by day 6 post-electroporation, 54%, T cells electroporated with either plasmid were only 1.4-2.6% viable. These data show that plasmid DNA was cytotoxic to T lymphocytes. In addition, these data show that DNA-mediated toxicity was not due to transposon element such as the ITR regions or the core insulators since the pmaxGFP™ plasmid are devoid of these elements and was also cytotoxic at the same DNA concentration. Both plasmids are approximately the same size, meaning that similar amounts of DNA were electroporated into the T cells.

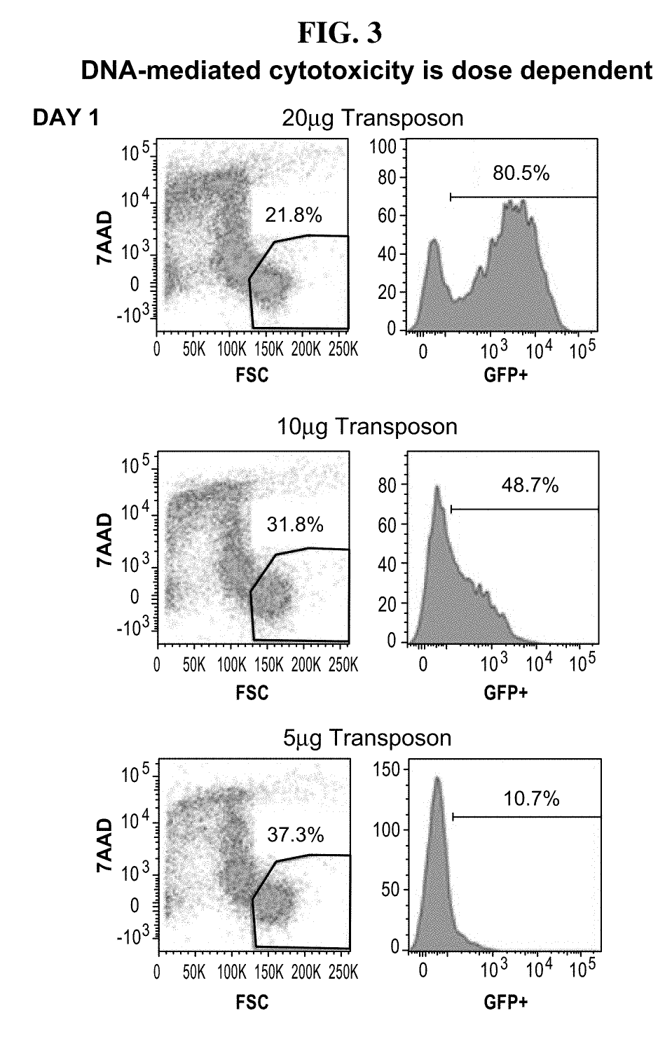

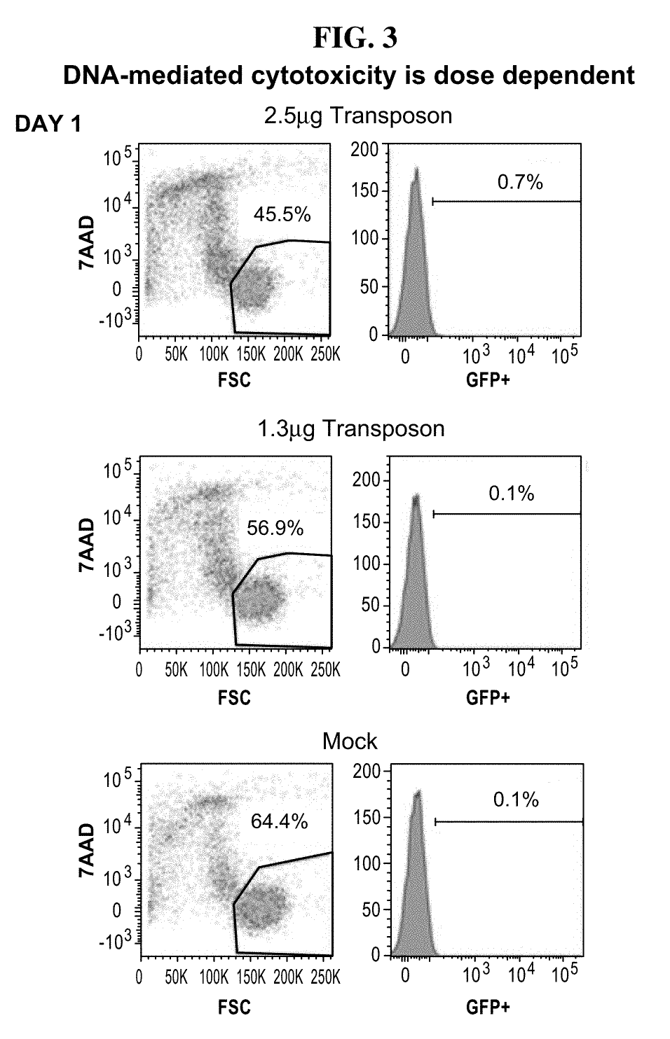

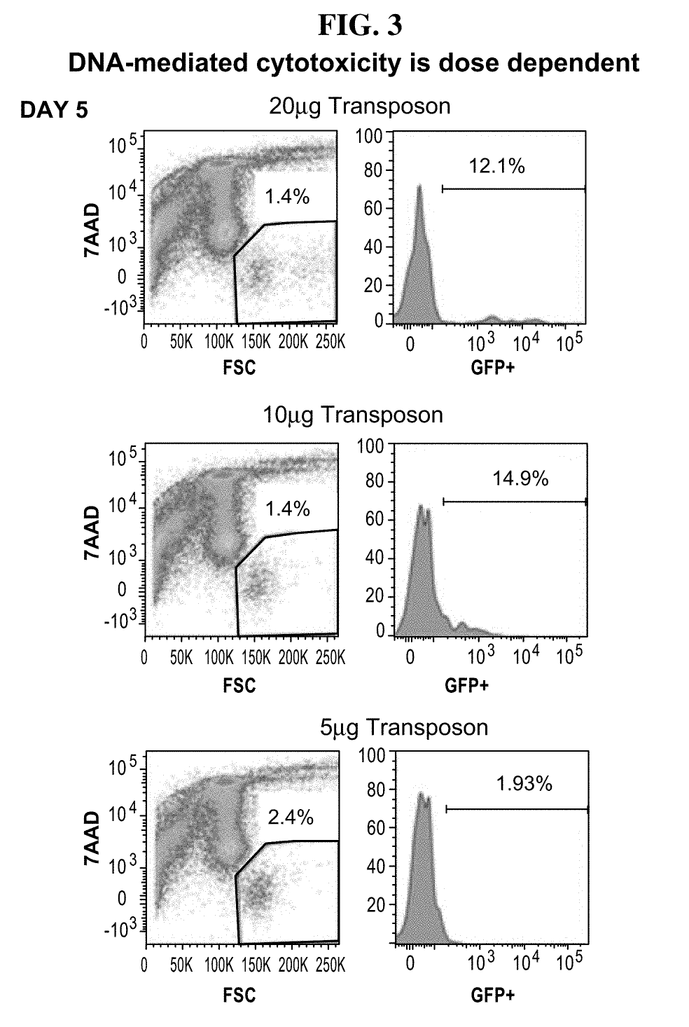

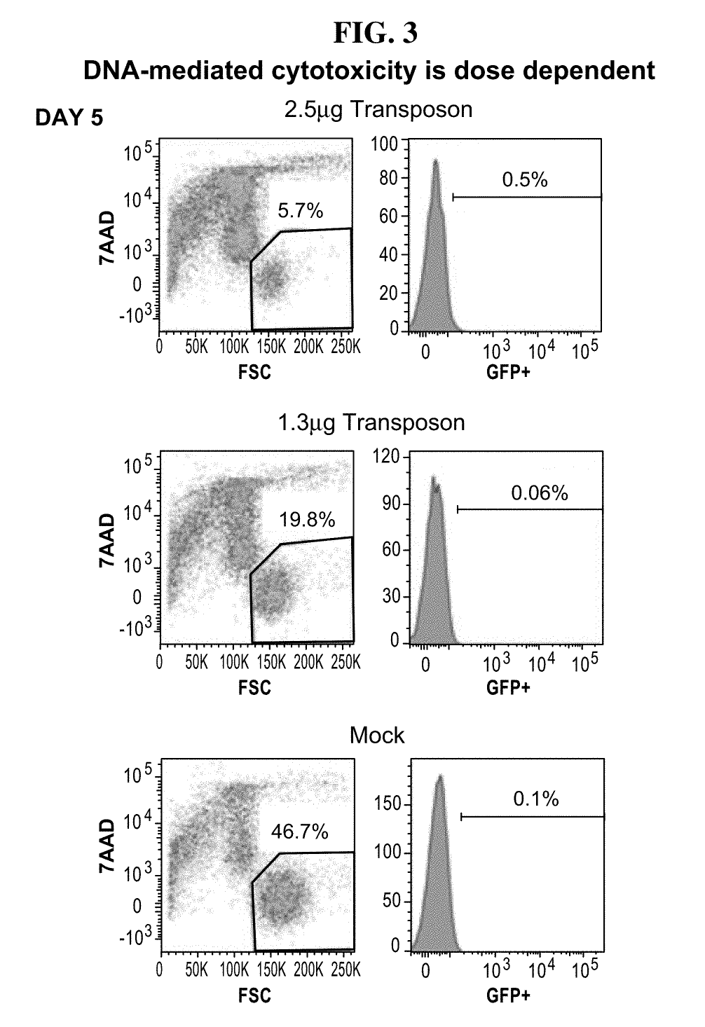

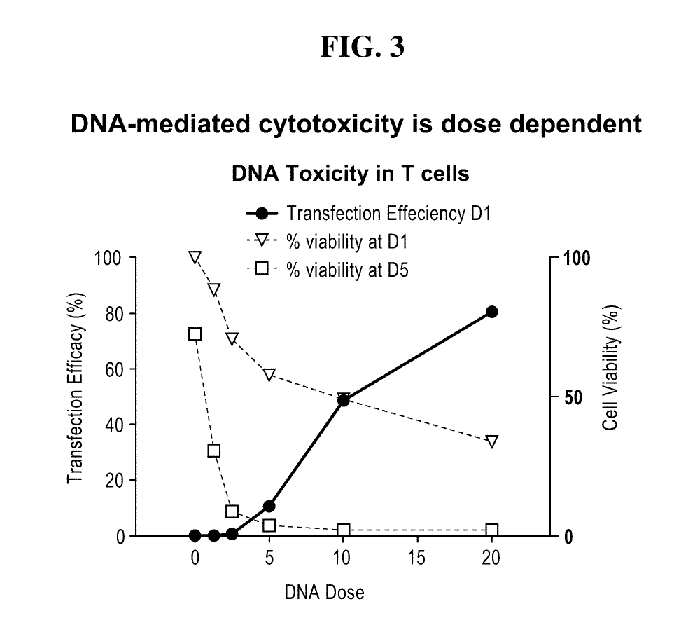

To test whether or not DNA-mediated toxicity in T cells was dose dependent, we performed a titration of our PB-GFP plasmid. FIG. 3 shows that as the dose of plasmid DNA added to the nucleofection reaction was increased incrementally (1.3, 2.5, 5.0, 10.0, and 20.0 μg of plasmid DNA), cell viability decreased as measured at both day 1 and 5 post-nucleofection. Even 1.3 μg of plasmid DNA was responsible for a 2.4-fold decrease in T cell viability by day 4.

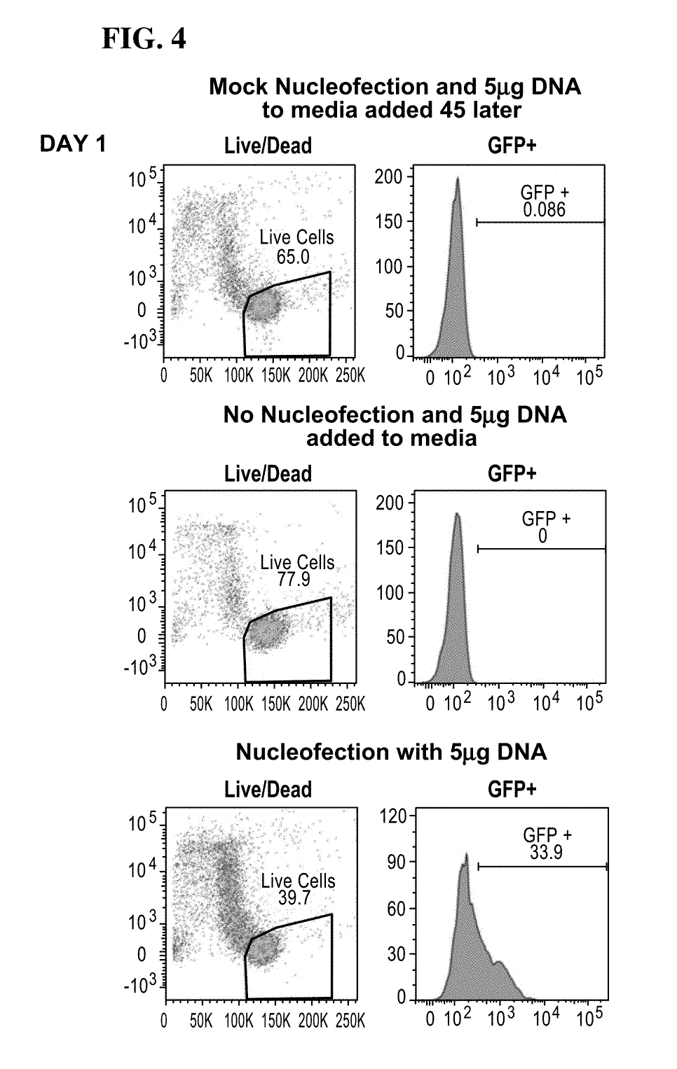

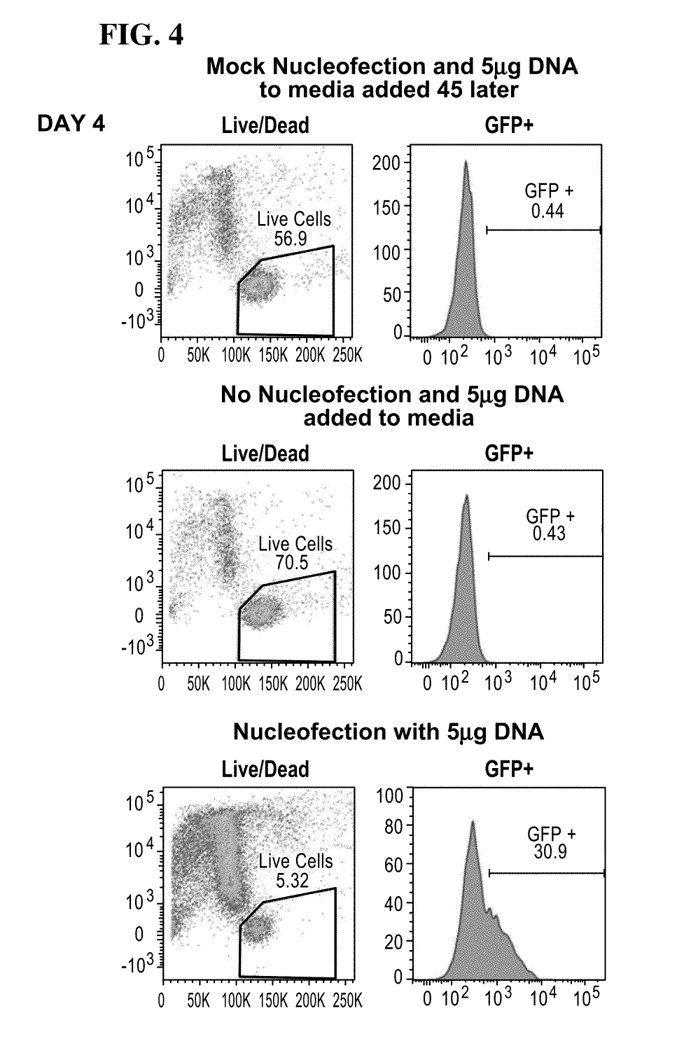

Since it was clear that plasmid DNA is toxic to T cells during nucleofection, we considered whether or not extracellular plasmid DNA was contributing to cell death. FIG. 4 shows that extracellular plasmid DNA was not cytotoxic to T cells. In that experiment, 5 μg of plasmid DNA was added to the cells 45 min post-electroporation and little cell death was observed at day 1 or day 4. Similarly, when 5 μg of plasmid DNA was added to the nucleofection reaction in the absence of electroporation, little cell death was observed. However, when the plasmid DNA was added before the electroporation reaction, the cells exhibited a 2.0-fold reduction in cell viability at day 1 and a 13.2-fold reduction at day 4.

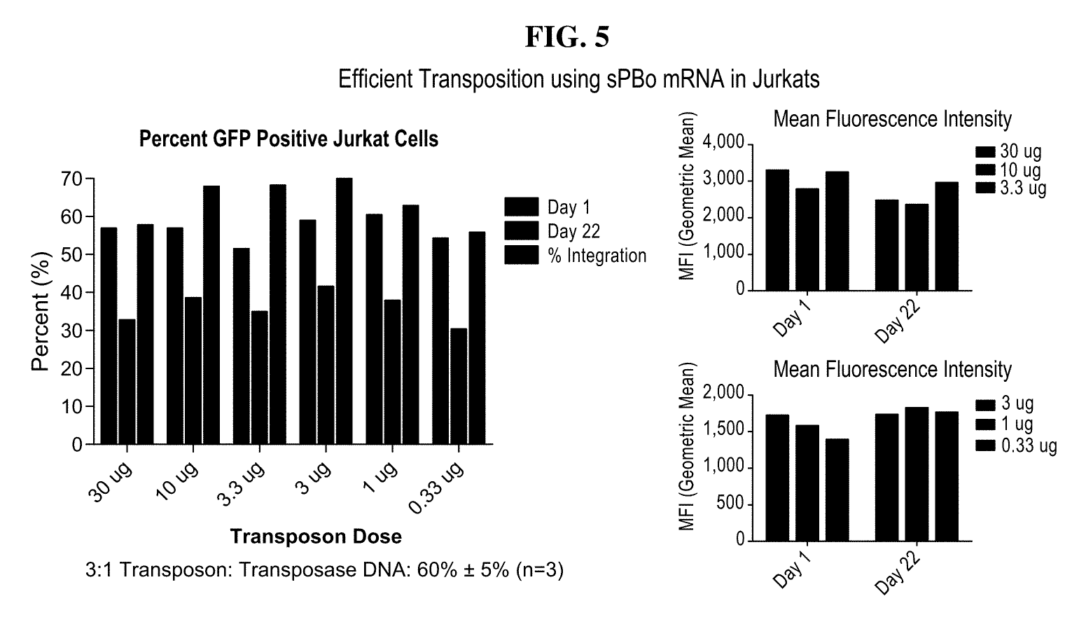

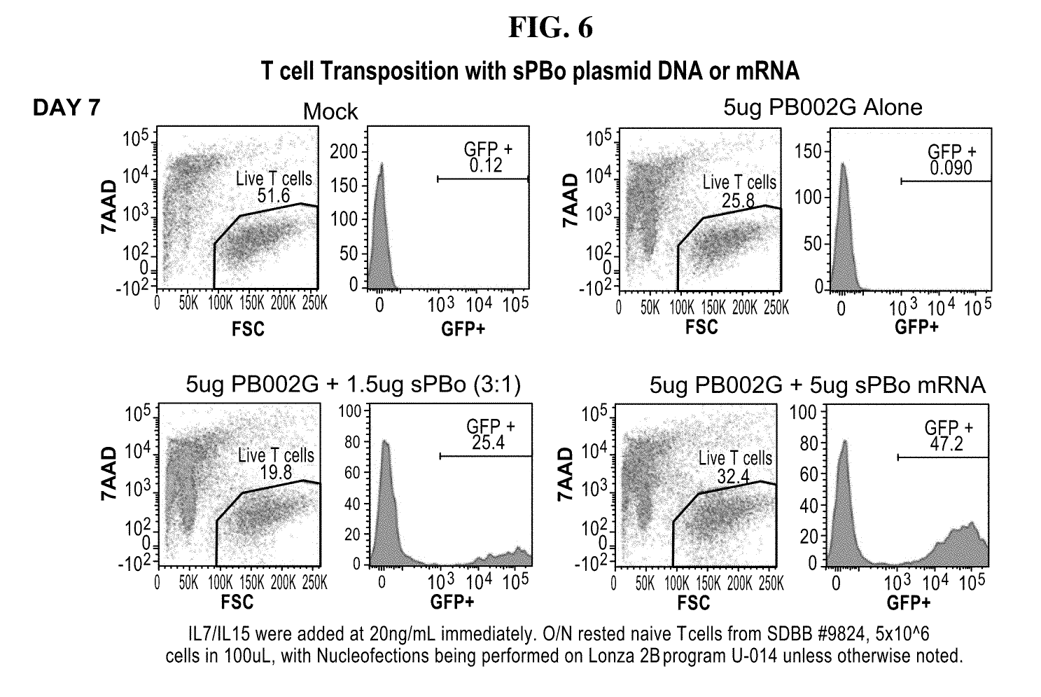

Since DNA-mediated toxicity is dose dependent, we next focused our attention on ways to reduce the total amount of DNA delivered to the T cells that is required for transposition. One relatively straightforward way of achieving this would be to deliver the transposase as encoded in mRNA instead of encoded in DNA. mRNA delivery to primary human T cells is very efficient, resulting in high transfection efficiency and high viability. We subcloned the Super piggyBac™ (sPBo) transposase enzyme into our in-house mRNA production vector and produced high quality sPBo mRNA. Co-delivery of PB-GFP transposon with various doses of sPBo mRNA (30, 10, 3.3, 3, 1, 0.33 μg mRNA) in Jurkat cells demonstrated strong transposition at all doses tested (FIG. 5). These data show that sPBo transposase can be delivered and are equally effective as either plasmid DNA or mRNA. In addition, that the amount of sPBo mRNA makes little difference in overall transposition efficiency in Jurkats, in either overall percentage of GFP+ cells or in the MFI of GFP expression. To see if this also holds true for T lymphocytes, we delivered PB-GFP with either sBPo plasmid DNA, at a 3:1 ratio, or 5 μg of sPBo mRNA. Seven (7) days following the nucleofection reaction and the addition of IL7 and IL15, GFP transposition was assessed. FIG. 6 shows that sPBo mRNA efficiently mediated transposition of the GFP transposon into T lymphocytes. Importantly, T cell viability was improved when co-delivering the sPBo as an mRNA as opposed to a pDNA; 32.4% versus 25.4%, respectively. These data suggest that co-delivery of sPBo as mRNA would be dose-sparing in the total amount of plasmid DNA being delivered to T cells and is thus less cytotoxic.

Since the current plasmid transposon also contains a backbone required for plasmid amplification in bacteria, it is possible to significantly reduce the total amount of DNA by excluding this sequence. This may be achieved by restriction digest of the plasmid transposon prior to the nucleofection reaction. In addition, this could be achieved by administering the transposon as a PCR product or as a Doggybone™ DNA, which is a double stranded DNA that is produced in vitro by a mechanism that excludes the initial backbone elements required for bacterial replication of the plasmid.

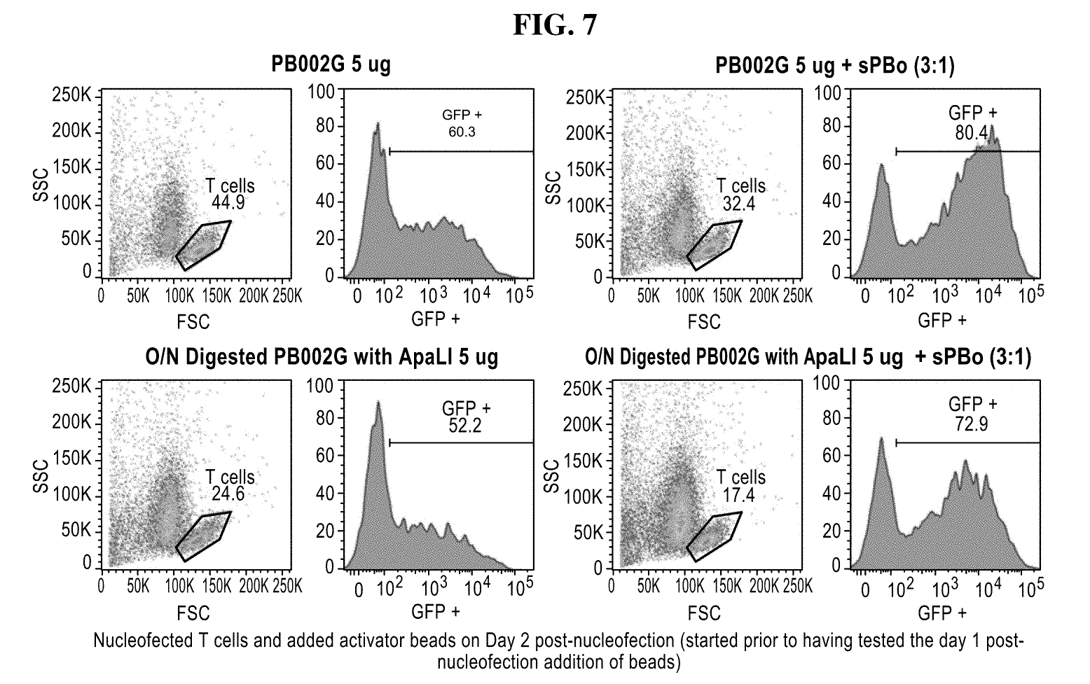

We performed a pilot experiment to see whether or not plasmid transposon needed to be circular, or if it could be delivered to the cell in a linear fashion. To test this, transposon was incubated overnight with a restriction enzyme (ApaLI) to linearize the plasmid. Either uncut or linearized plasmid was electroporated into primary T lymphocytes and GFP expression was assessed 2 days later. FIG. 7 shows that linearized plasmid was also efficiently delivered to the cell nucleus. These data demonstrate that linear transposon products can also be efficiently electroporated into primary human T cells.

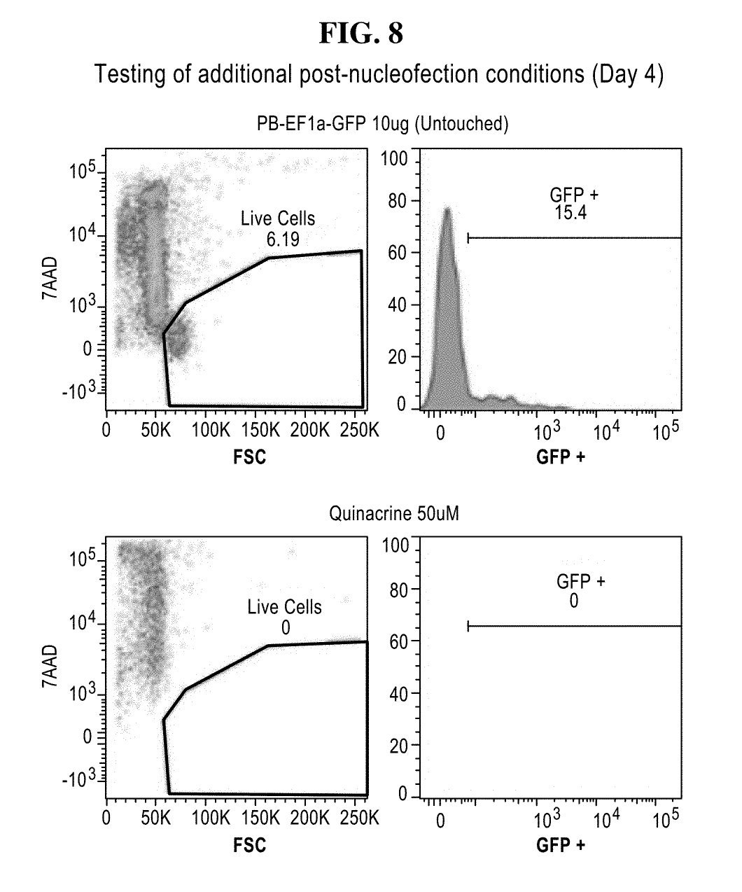

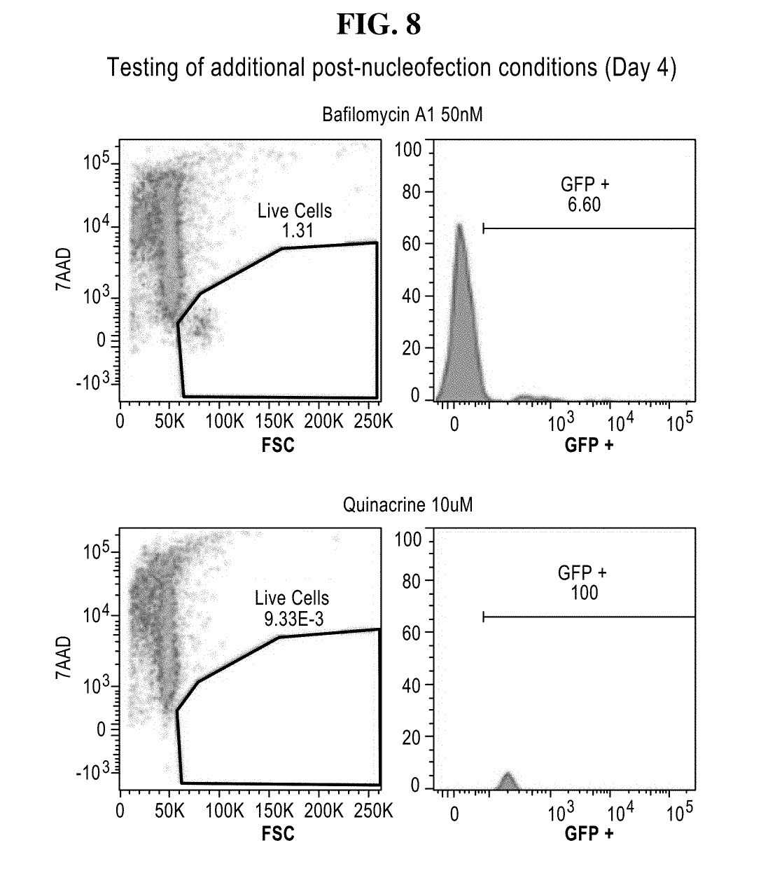

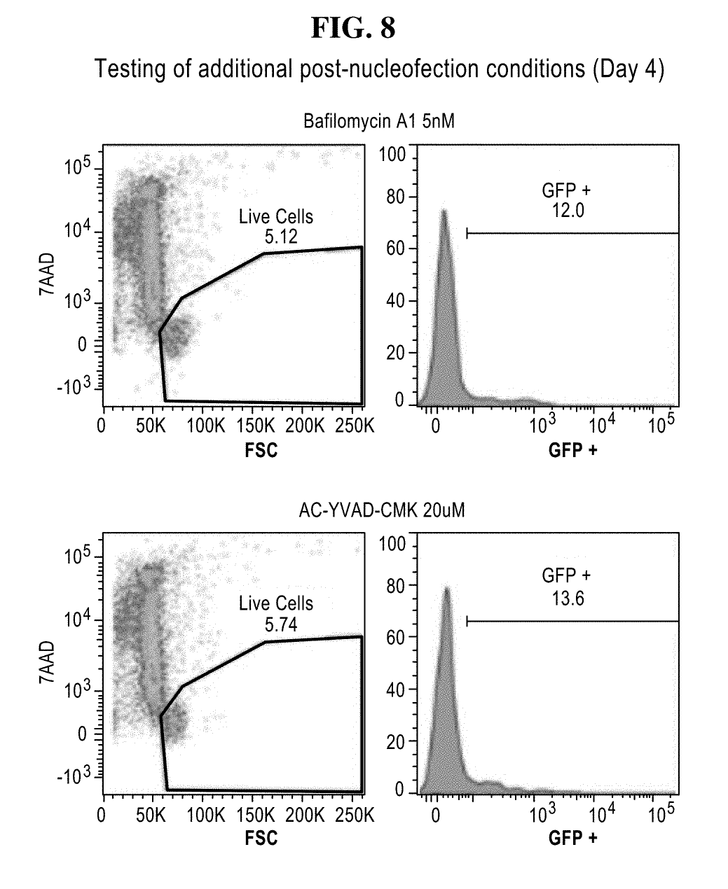

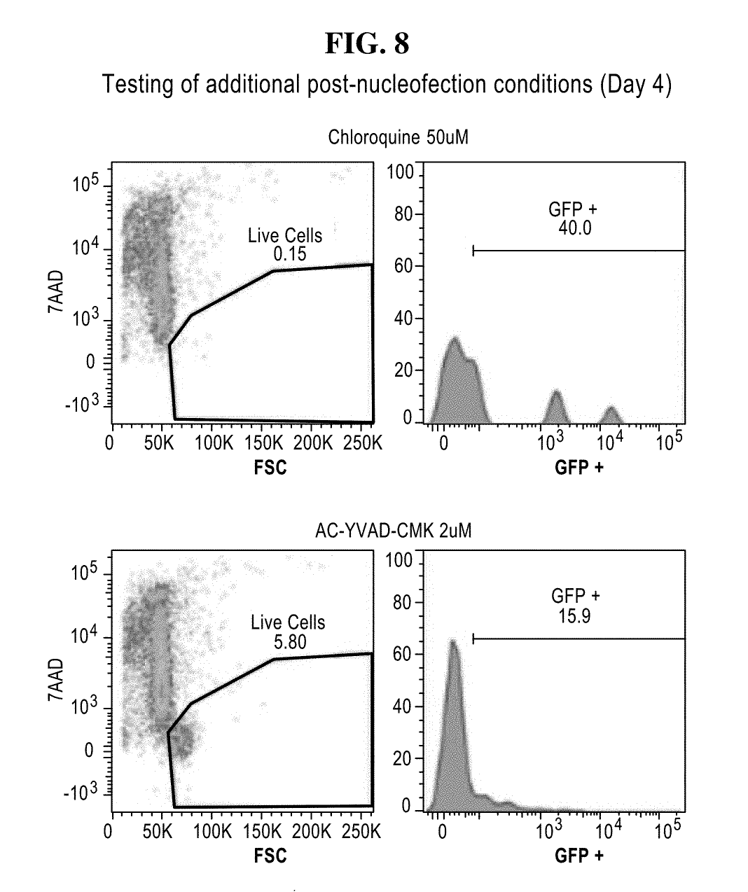

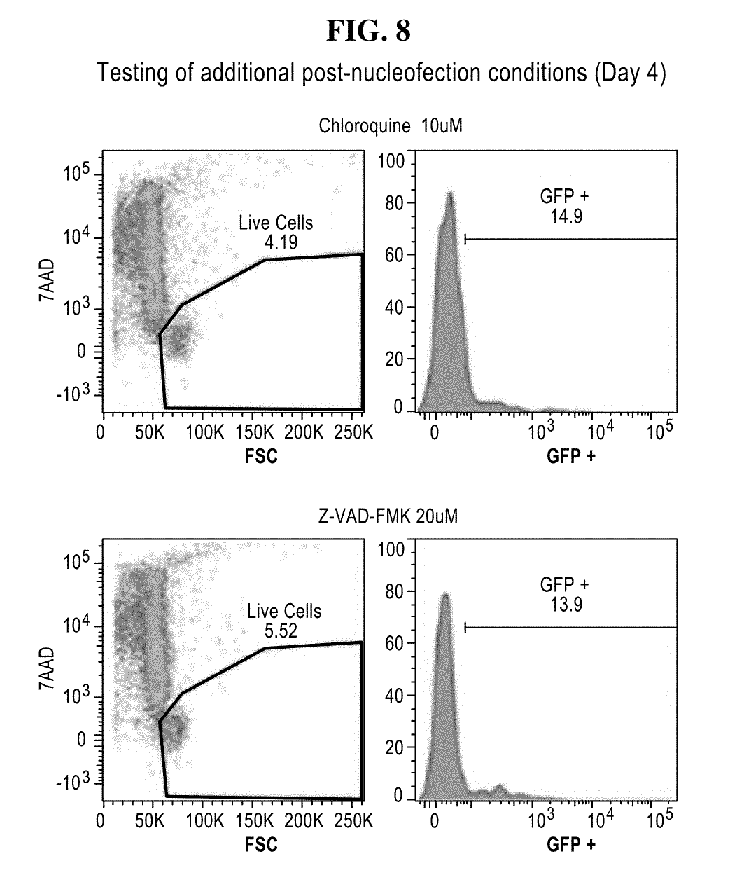

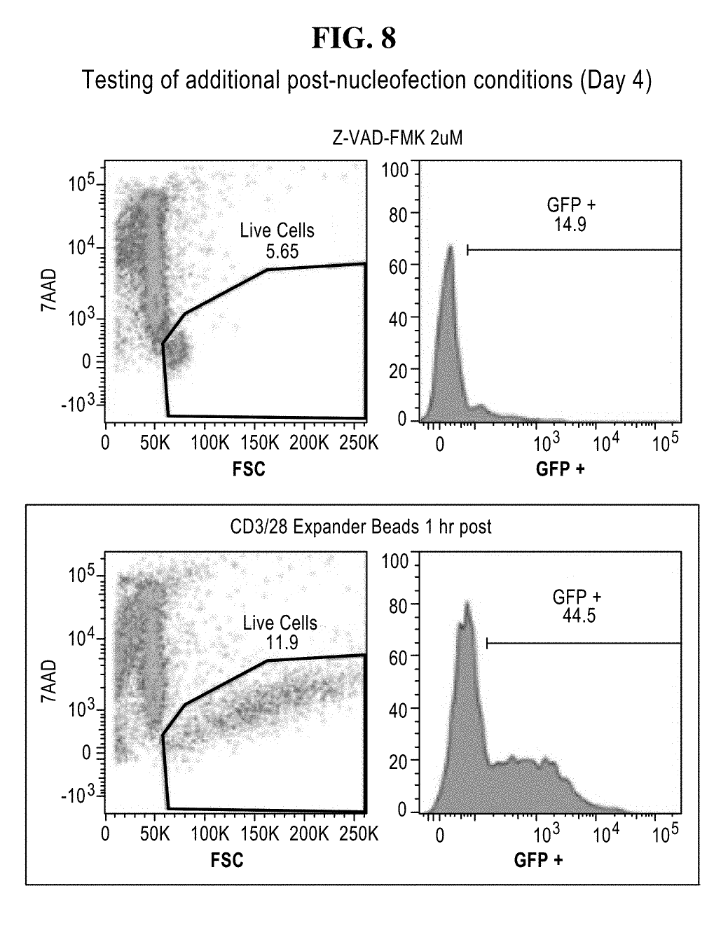

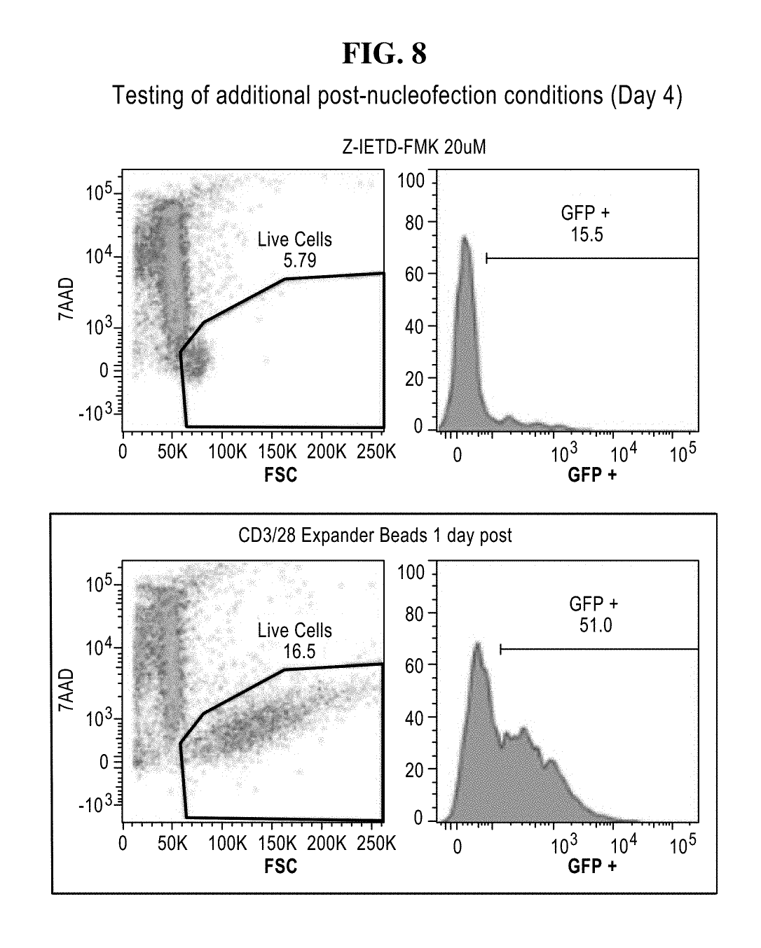

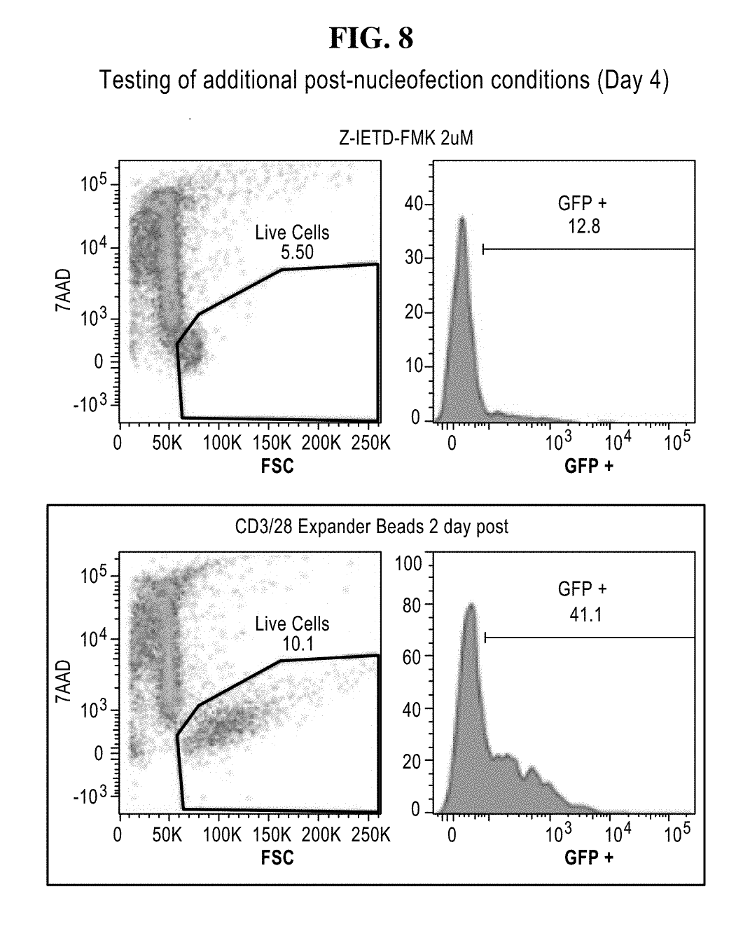

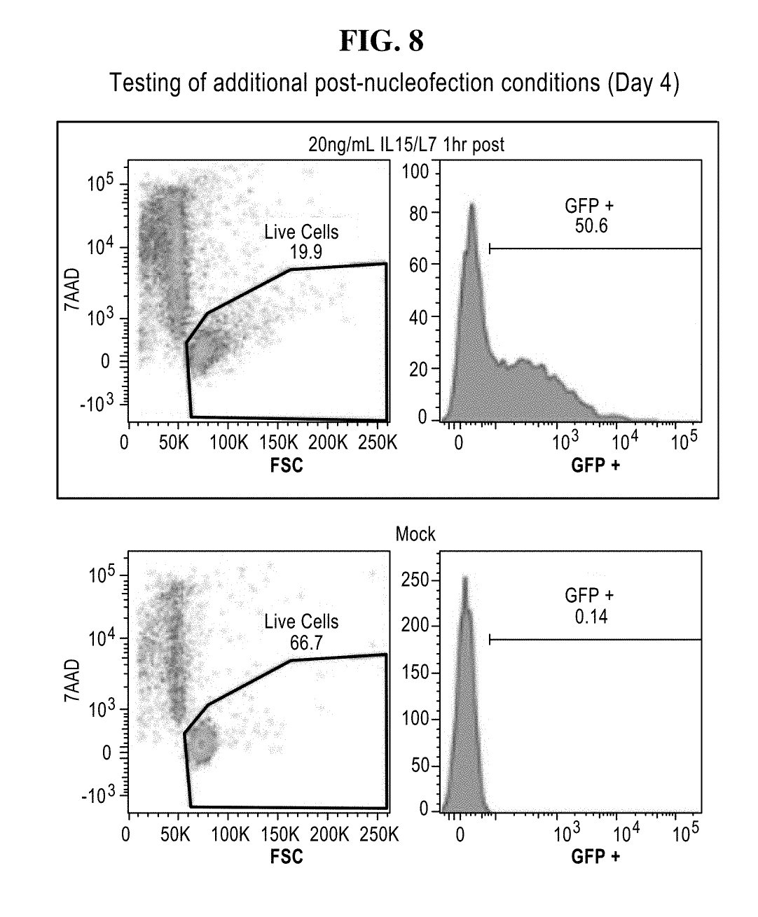

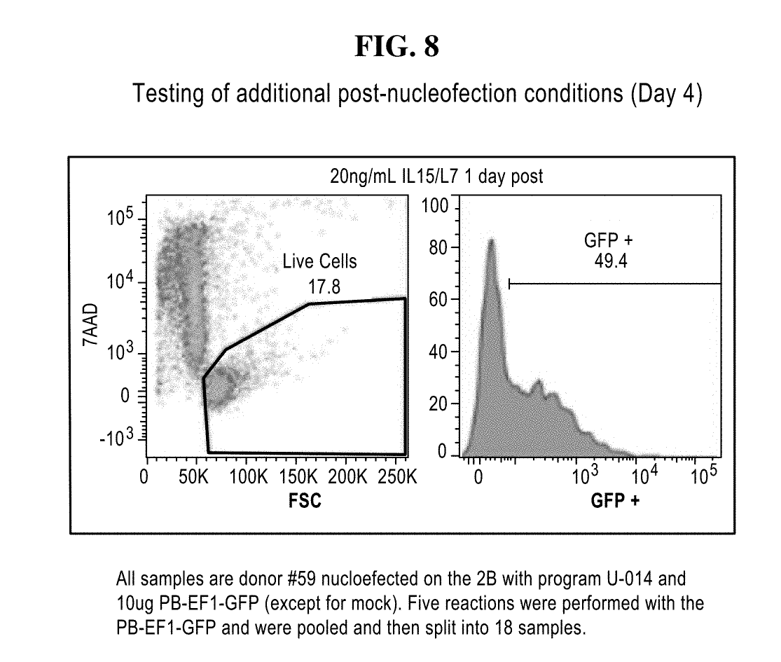

We show above that plasmid DNA is toxic in primary T lymphocytes, but we have observed that this toxic effect is not as dramatic in tumor cell lines and other transformed cells. Based upon this observation, we hypothesized that primary T lymphocytes may be refractory to plasmid DNA transfection due to heightened DNA sensing pathways, which would protect immune cells from infection by viruses and bacteria. If these data are a result of heightened DNA sensing mechanisms, then it may be possible to enhance plasmid transfection efficiency and/or cell viability by the addition of DNA sensing pathway inhibitors to the post-nucleofection reaction. Thus, we tested a number of different reagents that inhibited the TLR-9 pathway, caspase pathway, or those involved in cytoplasmic double stranded DNA sensing. These reagents include Bafilomycin Al, which is an autophagy inhibitor that interferes with endosomal acidification and blocks NFkB signaling by TLR9, Chloroquine, which is a TLR9 antagonist, Quinacrine, which is a TLR9 antagonist and a cGAS antagonist, AC-YVAD-CMK, which is a caspase 1 inhibitor targeting the AIM2 pathway, Z-VAD-FMK, which is a pan caspase inhibitor, Z-IETD-FMK, which is a caspase 8 inhibitor triggered by the TLR9 pathway. In addition, we also tested the stimulation of electroporated T cells by the addition of the cytokines IL7 and IL15, as well as the addition of anti-CD3 anti-CD28 Dynabeads® Human T-Expander CD3/CD28 beads. Results are displayed in FIG. 8. We found that few of the compounds or caspase inhibitors had any positive effect on cell viability at day 4 post-nucleofection at the doses tested. However, we acknowledge that further dosing studies may be required to better test these reagents. It may also be more effective to inhibit these pathways genetically. Two post-nucleofection conditions did enhance viability of the T cells. The addition of IL7 and IL15, whether they were added either 1 hour or 1 day following electroporation, enhanced viability over 3-fold when compared with introduction of the plasmid transposon alone without additional treatment. Furthermore, stimulation of the T cells post-nucleofection using either activator or expander beads also dramatically enhanced T cell viability; stimulation was better when the beads were added 1 hour or 1 day post-nucleofection as compared to adding them 2 days post. Lastly, we also tested ROCK inhibitor and the removal of dead cells from the culture using the Dead Cell Removal kit from Miltenyi, but saw no improvement in cell viability.

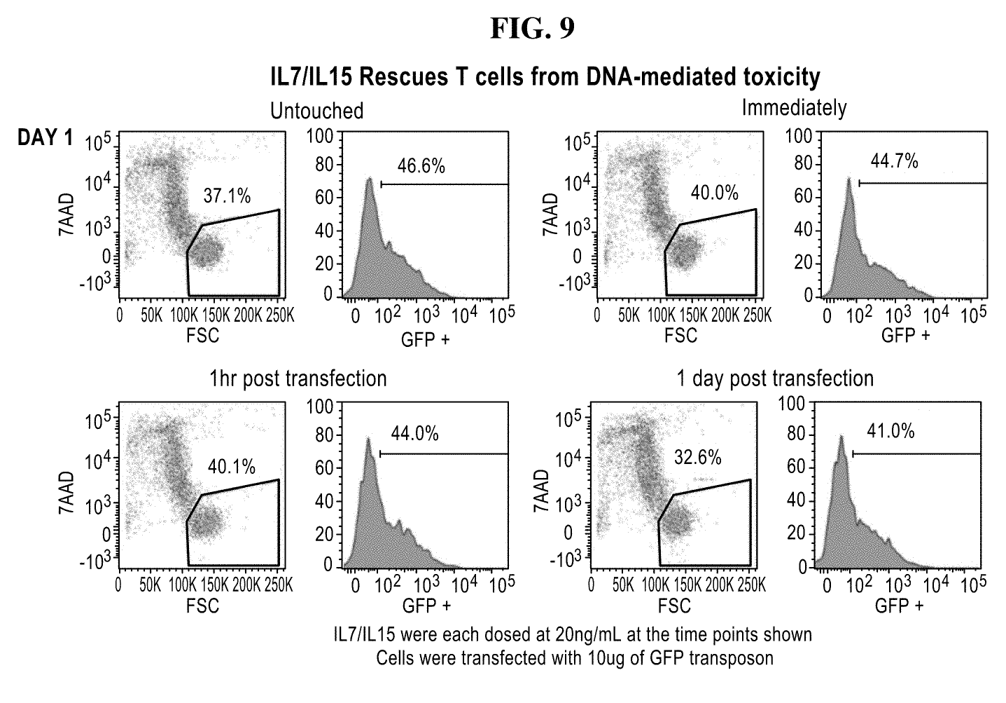

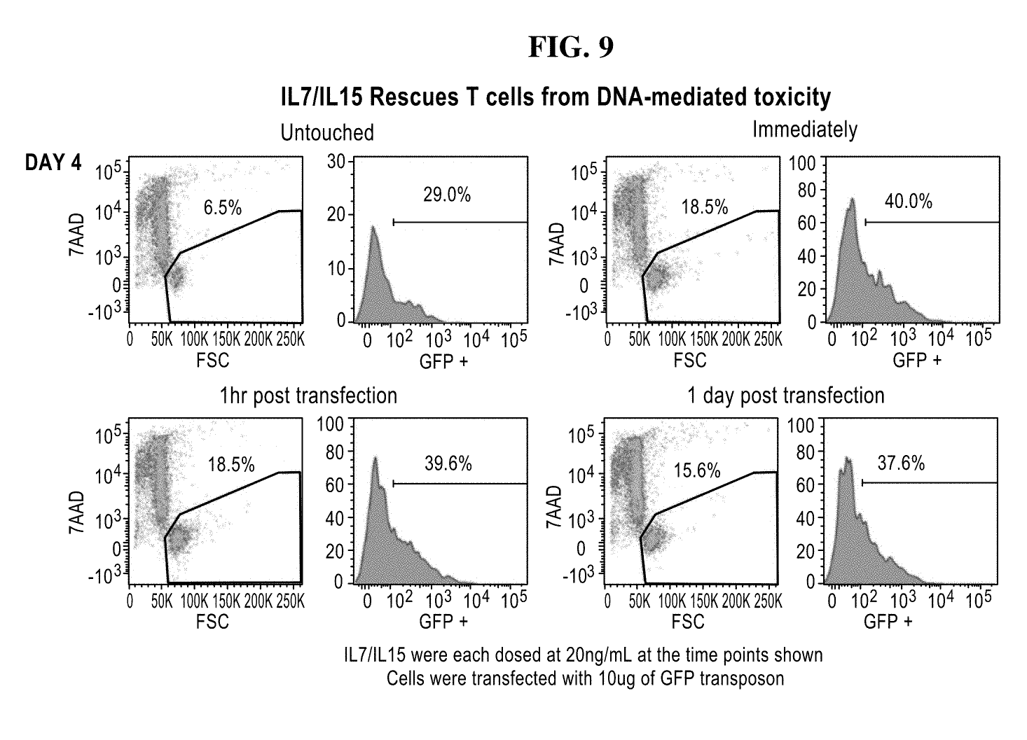

To further expand upon these findings demonstrating that stimulation of the T cells post-nucleofection improves viability, we repeated the study using the addition of the cytokine IL7 and IL15. FIG. 9 shows that the addition of these cytokines each at a dose of 20 ng/mL either immediately following nucleofection or up to 1 hour post enhanced cell viability up to 2.9-fold when compared to no treatment. Addition of these cytokines up to 1 day post-nucleofection also enhanced viability, but not as strong as the prior time points.

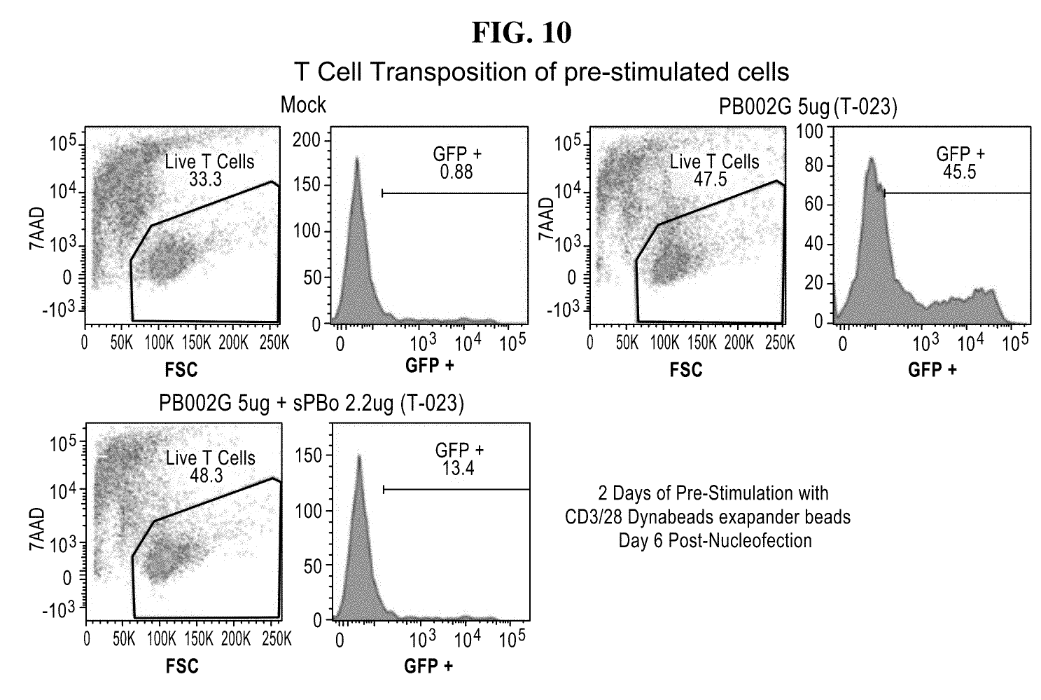

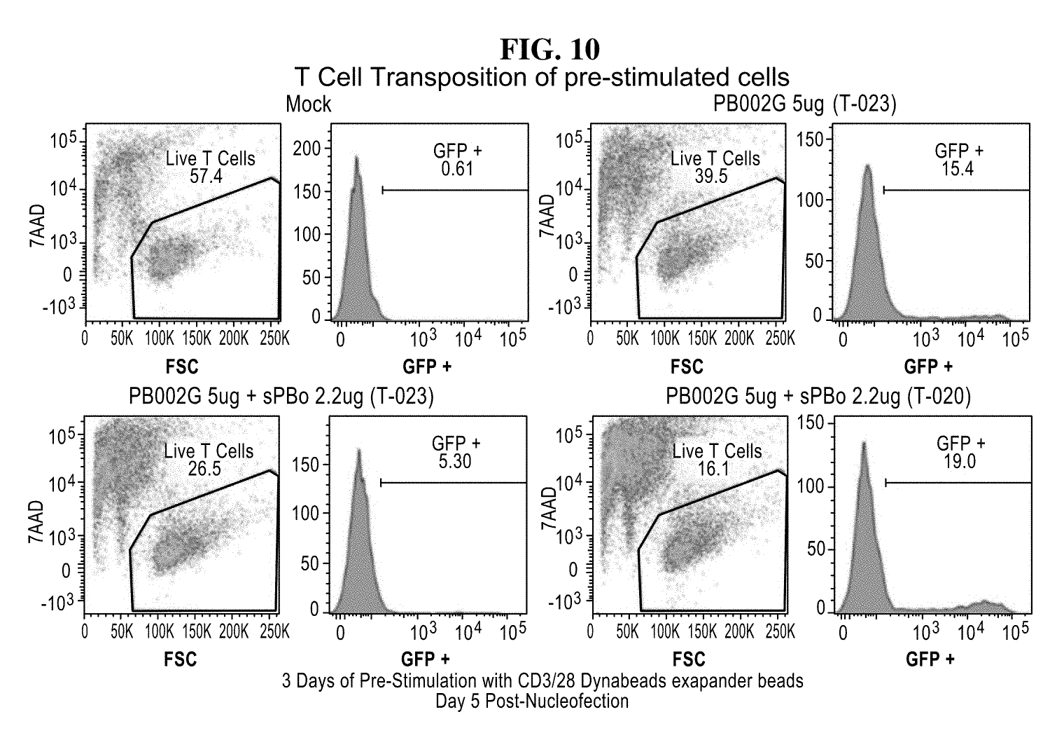

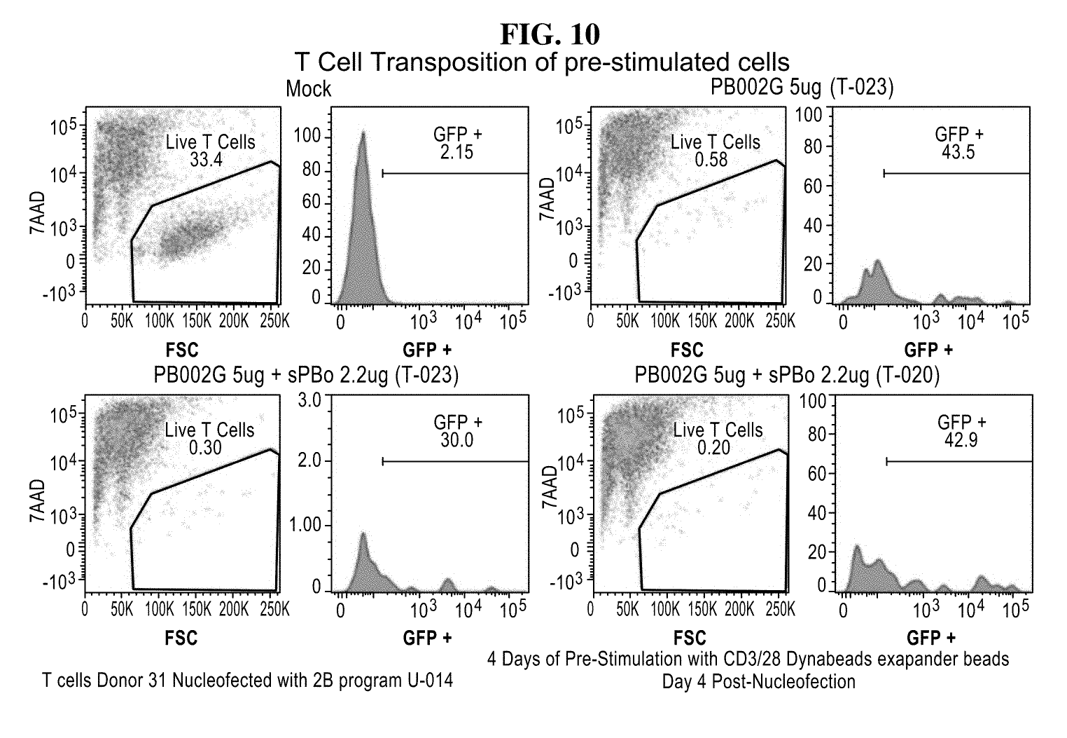

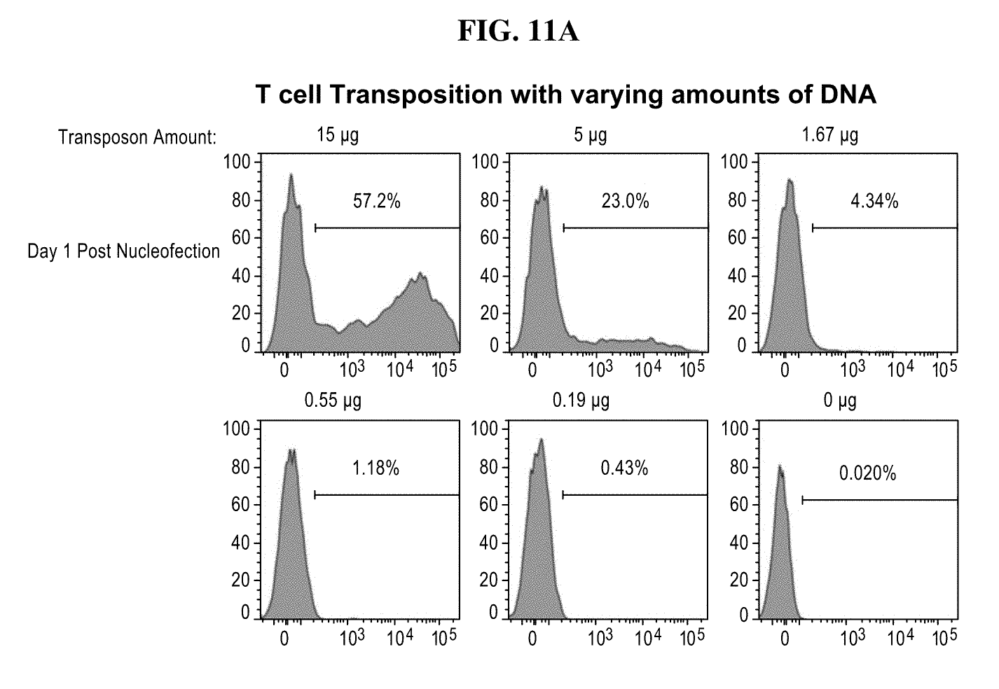

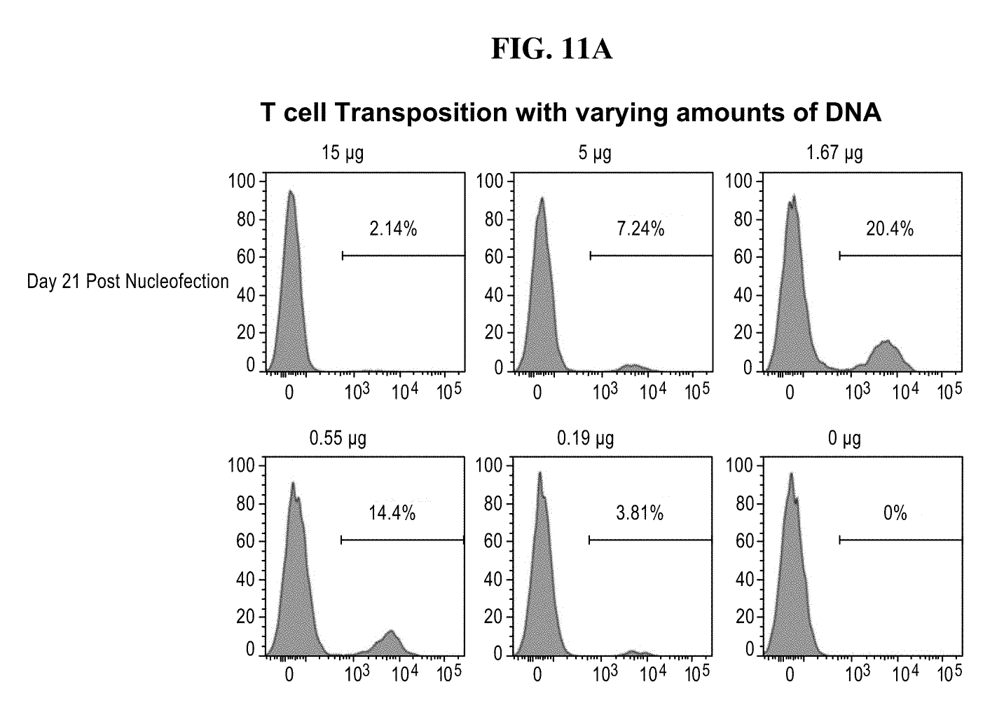

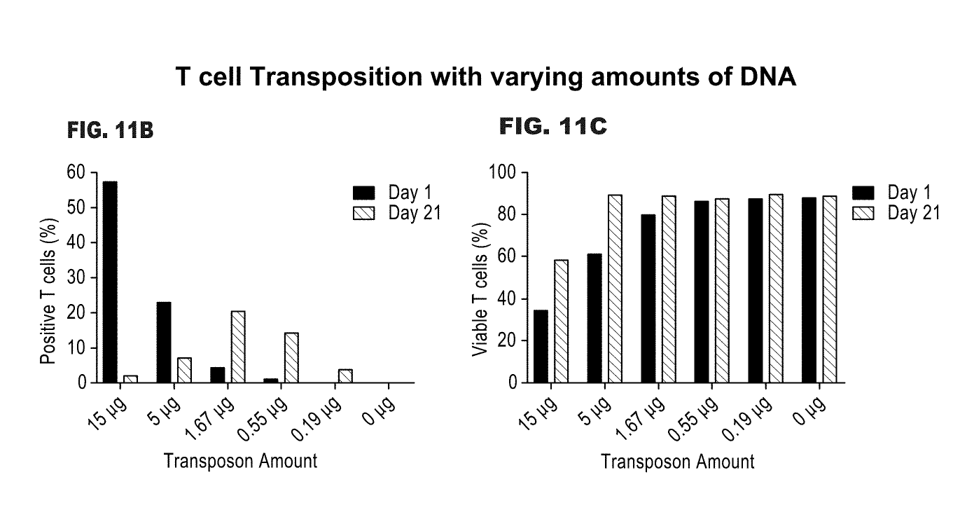

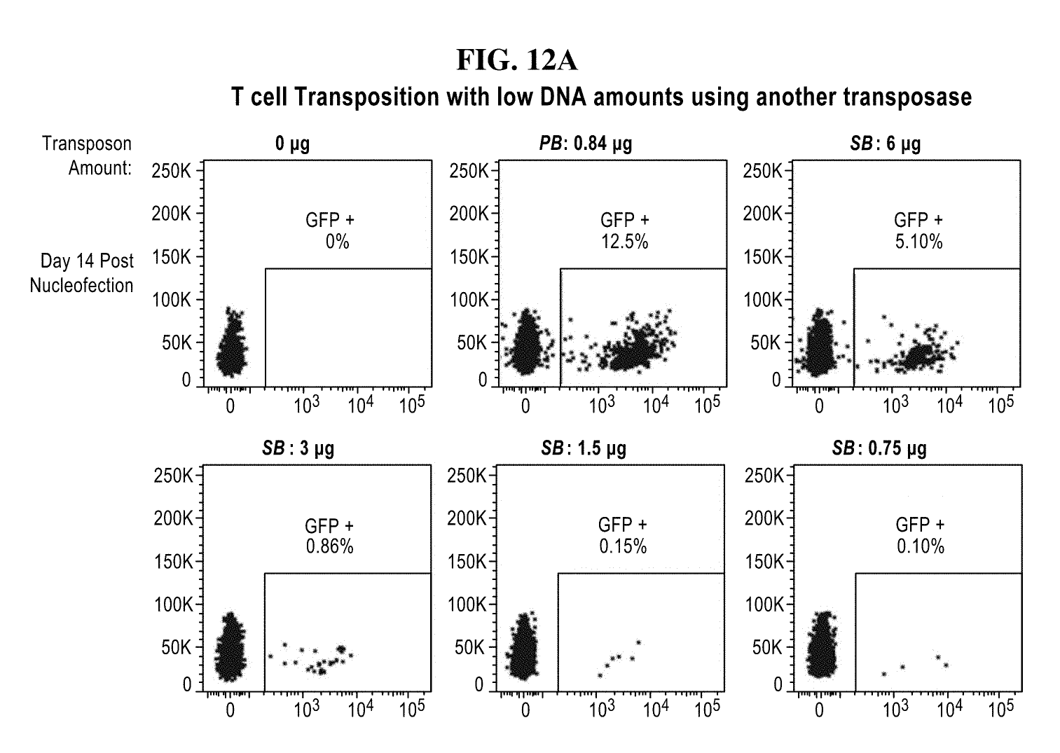

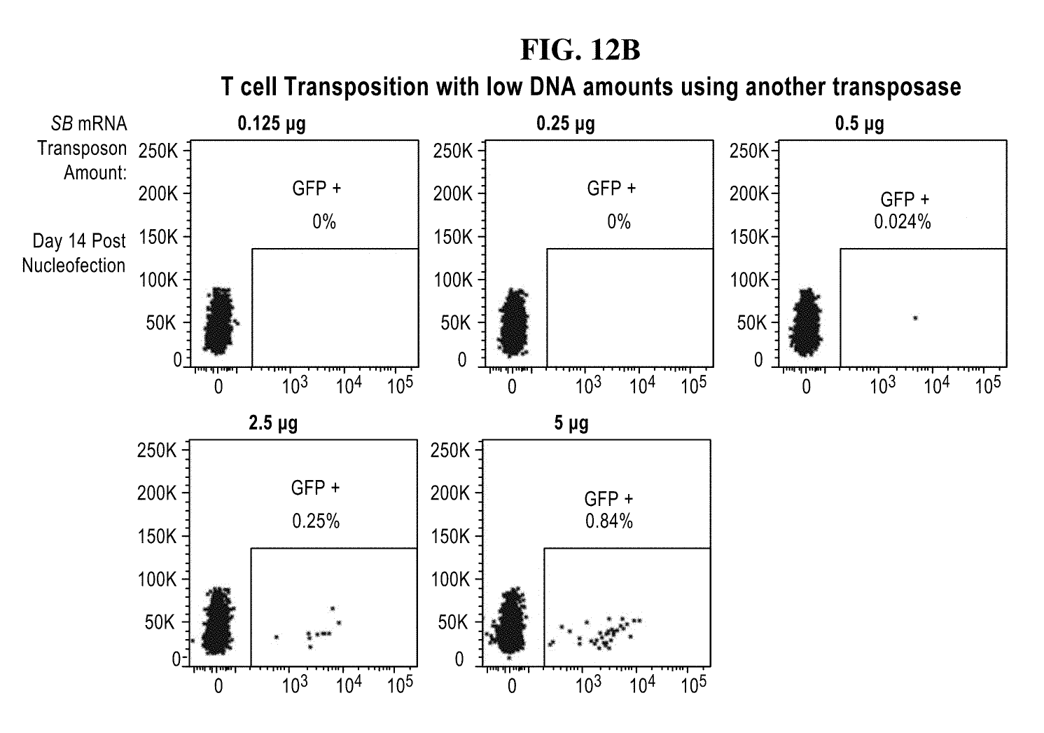

Since we found that immediate stimulation of the T cells post-nucleofection was able to increase cell viability, we hypothesized that stimulating the cells prior to nucleofection may also enhance viability and transfection efficiency. To test this, we stimulated primary T lymphocytes either 2, 3, or 4 days prior to transposon nucleofection. FIG. 10 shows that some level of transposition occurs when the transposon and the transposase are co-delivered after the T cells have been stimulated prior to the nucleofection reaction. The efficacy of pre-stimulation may be influenced by the kinetics of stimulation and may therefore be dependent upon the precise type of expander technology chosen.

1. A method for the ex-vivo genetic modification of an immune cell comprising delivering to the immune cell,
(a) a nucleic acid or amino acid sequence comprising a sequence encoding a transposase enzyme, wherein the nucleic acid sequence encoding the transposase enzyme is a DNA or an RNA sequence, and
(b) a recombinant and non-naturally occurring DNA sequence comprising a DNA sequence encoding a transposon, wherein the DNA sequence encoding the transposon comprises one or more of a circular DNA, a linear DNA, a linearized DNA, a single-stranded DNA, a double-stranded DNA, a plasmid DNA, and a minicircle DNA;
wherein the delivering step comprises electroporation or nucleofection of the immune cell,
wherein a total amount of DNA comprising an amount of a DNA sequence encoding the transposase enzyme and/or an amount of a DNA sequence encoding the transposon is equal to or less than 1.67 μg per 100 μL of an electroporation or nucleofection reaction, and
wherein a concentration of the total amount of DNA comprising the amount of the DNA sequence encoding the transposase enzyme and/or the amount of the DNA sequence encoding the transposon in the electroporation or nucleofection reaction is equal to or less than 16.7 μg/mL.

2-5. (canceled)

6. The method of claim 1, wherein the method further comprises the step of stimulating the immune cell with one or more cytokine(s).

7-10. (canceled)

11. The method of claim 1, wherein the immune cell is a T-lymphocyte.

12-16. (canceled)

17. The method of claim 1, wherein the transposase enzyme comprises an amino acid sequence at least 75% identical to:
(SEQ ID NO: 1)
MGSSLDDEHILSALLQSDDELVGEDSDSEVSDHVSEDDVQSDTEEAFIDE
VHEVQPTSSGSEILDEQNVIEQPGSSLASNRILTLPQRTIRGKNKHCWST
SKSTRRSRVSALNIVRSQRGPTRMCRNIYDPLLCFKLFFTDEIISEIVKW
TNAEISLKRRESMTSATFRDTNEDEIYAFFGILVMTAVRKDNHMSTDDLF
DRSLSMVYVSVMSRDRFDFLIRCLRMDDKSIRPTLRENDVFTPVRKIWDL
FIHQCIQNYTPGAHLTIDEQLLGFRGRCPFRVYIPNKPSKYGIKILMMCD
SGTKYMINGMPYLGRGTQTNGVPLGEYYVKELSKPVHGSCRNITCDNWFT
SIPLAKNLLQEPYKLTIVGTVRSNKREIPEVLKNSRSRPVGTSMFCFDGP
LTLVSYKPKPAKMVYLLSSCDEDASINESTGKPQMVMYYNQTKGGVDTLD
QMCSVMTCSRKTORWPMALLYGMINIACINSFIIYSHNVSSKGEKVQSPI
KFMRKLYMSLTSSFMRKRLEAPTLKRYLRDNISNILPKEVPGTSDDSTEE
PVMKKRTYCTYCPSKIRRKANASCKKCKKVICREHNIDMCQSCF.

18. The method of claim 1, wherein the transposase enzyme is a Sleeping Beauty transposase enzyme.

19. The method of claim 18, wherein the Sleeping Beauty transposase is a hyperactive Sleeping Beauty SB 100X transposase.

20. The method of claim 18, wherein the Sleeping Beauty transposase enzyme comprises an amino acid sequence at least 75% identical to:
(SEQ ID NO: 2)
MGKSKEISQDLRKKIVDLHKSGSSLGAISKRLKVPRSSVQTIVRKYKHHG
TTQPSYRSGRRRYLSPRDERTLVRKVQINPRTTAKDLVKMLEETGTKVSI
STVKRVLYRHNLKGRSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMRKENYVDILKQHLKTSVRKLKLGRKWVFQMDNDPKHT
SKVVAKWLKDNKVKVLEWPSQSPDLNPIENLWAELKKRVRARRPTNLTQL
HQLCQEEWAKIHPTYCGKLVEGYPKRLTQVKQFKGNATKY.

21-31. (canceled)

32. The method of claim 1, wherein the immune cell is obtained from a human.

33-34. (canceled)

35. The method of claim 1, wherein the recombinant and non-naturally occurring DNA sequence encoding a transposon further comprises a sequence encoding a chimeric antigen receptor or a portion thereof, wherein the portion of the sequence encoding a chimeric antigen receptor encodes an antigen recognition region, and wherein the antigen recognition region comprises a human or humanized antibody, an antibody mimetic, a protein scaffold or a fragment thereof.

36-40. (canceled)

41. The method of claim 35, wherein the antibody comprises or consists of a single-chain variable fragment (scFv), a VHH, a single domain antibody (sdAB), a small modular immunopharmaceutical (SMIP) molecule or a nanobody.

42-45. (canceled)

46. The method of claim 35, wherein the protein scaffold comprises or consists of Centyrin.

47-61. (canceled)

62. The method of claim 1,
(a) wherein the nucleic acid sequence encoding the transposase enzyme is a DNA or an RNA sequence,
(b) wherein a total amount of DNA comprising an amount of the DNA sequence encoding the transposase enzyme and/or an amount of the DNA sequence encoding the transposon is equal to or less than 0.55 μg per 100 μL of an electroporation or nucleofection reaction, and
wherein a concentration of the total amount of DNA comprising the amount of the DNA sequence encoding the transposase enzyme and/or the amount of the DNA sequence encoding the transposon in the electroporation or nucleofection reaction is equal to or less than 5.5 μg/mL.

63. (canceled)

64. The method of claim 1,
(a) wherein the nucleic acid sequence encoding the transposase enzyme is a DNA or an RNA sequence,
(b) wherein a total amount of DNA comprising an amount of the DNA sequence encoding the transposase enzyme and/or an amount of the DNA sequence encoding the transposon is equal to or less than 0.19 μg per 100 μL of an electroporation or nucleofection reaction, and
wherein a concentration of the total amount of DNA comprising the amount of the DNA sequence encoding the transposase enzyme and/or the amount of the DNA sequence encoding the transposon in the electroporation or nucleofection reaction is equal to or less than 1.9 μg/mL.

65. (canceled)

66. The method of claim 1,
(a) wherein the nucleic acid sequence encoding the transposase enzyme is a DNA or an RNA sequence,
(b) wherein a total amount of DNA comprising an amount of the DNA sequence encoding the transposase enzyme and/or an amount of the DNA sequence encoding the transposon is equal to or less than 0.1 μg per 100 μL of an electroporation or nucleofection reaction, and
wherein a concentration of the total amount of DNA comprising the amount of the DNA sequence encoding the transposase enzyme and/or the amount of the DNA sequence encoding the transposon in the electroporation or nucleofection reaction is equal to or less than 1.0 μg/mL.

67-109. (canceled)

110. The method of claim 1, wherein the immune cell is a natural killer (NK) cell.

111. The method of claim 1, wherein the immune cell is a cytokine-induced killer (CIK) cell.

112. The method of claim 1, wherein the immune cell is a natural killer T (NKT) cell.

113. The method of claim 32, wherein the immune cell is derived from a primary cell, a cultured cell or cell line, an embryonic or adult stem cell, an induced pluripotent stem cell or a transdifferentiated cell.

114. The method of claim 32, wherein the immune cell is derived from a cell or cell line, and wherein the cell or cell line has been modified to suppress one or more apoptotic pathways.

115. The method of claim 32, wherein the immune cell is derived from a cell or cell line, and wherein the cell or cell line has been modified to allogeneic.

116. The method of claim 35, wherein the chimeric antigen receptor (CAR) comprises
(a) an ectodomain comprising the antigen recognition region,
(b) a transmembrane domain, and
(c) an endodomain comprising at least one costimulatory domain.

117. The method of claim 116, wherein the ectodomain further comprises a signal peptide.

118. The method of claim 117, wherein the signal peptide comprises a sequence encoding a human CD2, CD3δ, CD3ε, CD3γ, CD3ζ, CD4, CD8a, CD19, CD28, 4-1BB or GM-CSFR signal peptide.

119. The method of claim 116, wherein the ectodomain further comprises a hinge between the antigen recognition region and the transmembrane domain.

120. The method of claim 119, wherein the hinge comprises a sequence derived from a human CD8a, IgG4, and/or CD4 sequence.

121. The method of claim 116, wherein the transmembrane comprises a sequence encoding a human CD2, CD3δ, CD3ε, CD3γ, CD3ζ, CD4, CD8a, CD19, CD28, 4-1BB or GM-CSFR transmembrane domain.

122. The method of claim 116, wherein the endodomain comprises a sequence encoding a human CD3ζ endodomain.

123. The method of claim 116, wherein the at least one costimulatory domain comprises a human 4-1BB, CD28, CD40, ICOS, MyD88, OX-40 intracellular segment, or any combination thereof.

124. The method of claim 116, wherein the at least one costimulatory domain comprises a CD28 and/or a 4-1BB costimulatory domain.

125. The method of claim 35, wherein the single domain antibody comprises one variable domain (VH) of a heavy-chain antibody or of a common IgG.
